(19) United States
(12) Patent Application Publication
(10) Pub. No.: US 2009/0048537 A1
(43) Pub. Date: Feb. 19, 2009

(54) Lubricious coatings

(75) Inventors: Margaret Lydon, North Chili, NY (US); Rui Avelar, Vancouver (CA); Richard J. Whitbourne, Rochester, NY (US); Donald M. Copenhagen, Bloomfield, NY (US); Kristy Jackson, Rochester, NY (US); Martin Jay Kozlowski, Kutztown, PA (US)

Correspondence Address: STEPTOE & JOHNSON LLP, 1330 CONNECTICUT AVENUE, N.W., WASHINGTON, DC, 20036, US

(73) Assignee: ANGIOTECH BIOCOATINGS CORP., Henrietta, NY (US)

(21) Appl. No.: 12/162,556

(22) PCT Filed: Jan. 31, 2007
(86) PCT No.: PCT/US2007/002434

§ 371 Date: Oct. 17, 2008

Related U.S. Application Data
(60) Provisional application No. 60/763,361, filed on Jan. 31, 2006.
(60) Provisional application No. 60/763,920, filed on Feb. 1, 2006.
(60) Provisional application No. 60/835,086, filed on Aug. 3, 2006.

(51) Int. Cl. A61M 25/16; B05D 3/00; A61B 17/3211; A61M 25/09

(52) U.S. Cl. 600/585; 427/2.1; 606/167; 604/265

(57) ABSTRACT

Medical devices can include a lubricious coating that is slippery when wet.

### CLAIM OF PRIORITY

This application claims priority to provisional U.S. Patent Application No. 60/763,361, titled “Lubricious Coating for Surgical Instruments,” filed Jan. 31, 2006, to provisional U.S. Patent Application No. 60/763,920, titled “Lubricious Echogenic Coating for Surgical Instruments,” filed Feb. 1, 2006, and to provisional U.S. Patent Application No. 60/835,086, titled “Lubricious Coating for Surgical Instruments,” filed Aug. 3, 2006, each of which is incorporated by reference in its entirety.

### TECHNICAL FIELD

The invention relates to lubricious coatings.

### BACKGROUND

A health care provider seeks to carry out operations with a high degree of accuracy and precision so as to minimize unnecessary trauma and/or damage to a patient. The health care provider uses a variety of instruments to perform surgical procedures, including, for example, dissection, curetting, suturing, and cutting with scissors or a scalpel, and many others known in the art. Because the instrument contacts the patient, the properties of the instrument can influence the extent to which a patient's tissues suffer undesired trauma or damage. The health care provider also contacts the instrument, and so the properties of the instrument can influence how effectively the health care provider can use the instrument.

### SUMMARY

Medical devices can be coated with hydrophilic, lubricant, and optionally echogenic coatings. Such coated devices can thus be slippery when wet, yet non-slippery when dry, and optionally echogenic. Generally, a medical device can have a surface coated with a performance enhancing coating system including one or more layers that render the surface more lubricious and biocompatible. The coated devices can be provided in a dry state, so as to be non-slippery for ease of handling and preparation. Once the device is wetted, before or during a medical procedure, it can become slippery so as to protect the patient and can reduce friction and damage to surrounding tissue. A device that has an echogenic coating can be readily distinguished from surrounding tissue or fluid when observed by ultrasound imaging.

Medical devices may include appropriate dye components to make them optically visible during surgical procedures. The coating can be employed to reduce the coefficient of friction of instruments including, for example, knives, scalpels, rongeurs, dissectors, scissors, needle drivers, suture holders, curettes, electrodes, probes, forceps, aneurysm clip applicators, and the like. The coatings can enhance the ultrasound visibility of surfaces of needles, catheters, and laparoscopic devices, such as intravascular retrieval snares or baskets.

In one aspect, a medical device includes a surface coated with a first layer proximal to the surface, the first layer including an ethylene/acrylic acid copolymer. The first layer can further include an epoxy resin. The device can be a blade device. A second layer can be coated on the first layer, the second layer including an aromatic polycarbonate based polyurethane. The second layer can further include a cellulose based polymer. A third layer can be coated on the second layer, the third layer including a polyvinyl pyrrolidone. The third layer can further include a cellulose based polymer.

The first layer can be coated on the surface by dip-coating with a solution comprising the ethylene/acrylic acid copolymer, an epoxy resin, tetrahydrofuran, dimethyl acetamide, anisole, and xylenes, and drying.

The second layer can be coated on the first layer by dip-coating with a solution comprising an aromatic polycarbonate based polyurethane, a nitrocellulose, dimethylacetamide, anisole, and an organic solvent, such as a mixture of one or more of methyl ethyl ketone and n-butanol, and drying.

The third layer can be coated on the second layer by dip-coating with a solution including a polyvinylpyrrolidone, a nitrocellulose, 4-butyrolactone, and an organic solvent, such as a mixture of one or more of ethanol, benzyl alcohol, cyclohexanone, and isopropanol, and drying.

In another aspect, a medical device includes a surface coated with a first layer proximal to the surface, the first layer including a cellulose based polymer, a urethane, a melamine resin, and a cross-linkable acrylic resin. The device can be a guidewire. A second layer can be coated on the first layer, the second layer including a nitrocellulose, a polyvinylpyrollidone, and a plasticizer. The plasticizer can be a poly(alkylene oxide). The urethane can be Tycel 7000. The melamine resin can be Cymel 248-8. The cross-linkable acrylic resin can be Paraloid AT-746. The polyvinylpyrollidone can be PVP K90. The poly(alkylene oxide) can be a polyethylene glycol 400.

In another aspect, a medical device includes a surface coated with a first layer proximal to the surface, the first layer being coated on the surface by contacting the surface with a solution comprising ethanol, benzyl alcohol, cyclohexanone, tetrahydrofuran, a nitrocellulose, 4-butyrlactone, and a polyvinylpyrrolidone, and drying. The device can be a catheter. The surface can be a thermoplastic polyurethane elastomer. The thermoplastic polyurethane elastomer can be a pellethane.

In another aspect, a medical device includes a surface coating bonded to a surface of the device, wherein the coating swells when exposed to body fluids; wherein the coating provides a substantial reduction in surface friction after swelling. The coating can include multiple layers. The coating can include a polyvinylpyrrolidone, a polyvinylpyrrolidone/vinyl acetate copolymer, and a polyethylene glycol. The coating can include a cellulose ester, a polyvinyl chloride, an acrylic polymer or copolymer, a polyurethane, a polyamide polymer, a polyimide polymer, or an epoxy resin. The polyvinylpyrrolidone can have a molecular weight of at least 80 kDa, or a molecular weight in the range of 90 kDa to 1,200 kDa. The cellulose ester can be a nitrocellulose.

The medical device can be a catheter, an arterial catheter, a short-term central venous catheter, a long-term central venous catheter, a peripheral venous catheter, a vascular port catheter, a dialysis device, a guide wire, an introducer, a knife, a needle, an amniocentesis needle, a biopsy needle, an infusion needle, an introducer needle, a suture needle, an obdurator, a pacemaker, a pacemaker lead, a penile prosthesis, a scalpel, a shunt, an arteriovenous shunt, a hydrocephalus shunt, a stent, a biliary stent, a coronary stent, a neurological stent, a urological stent, a vascular stent, a syringe, a trocar, a tube, a drain tube, an endotracheal tube, a gastroenteric tube, a nasogastric tube, an intermittent urinary catheter, a Foley catheter, a long-term urinary device, a tissue bonding urinary device, a urinary dilator, a urinary sphincter, a urethral inserts or a wound drain.

The surface coating can include a first layer proximal to the device surface, the first layer including an ethylene/acrylic acid copolymer. The first layer can further include an epoxy resin.

The surface of the device can include a metal or metal alloy. The metal or metal alloy can be gold, nitinol, nickel, platinum, stainless steel, tantalum, or titanium. The surface of the device can include a polymer or copolymer selected from a silicone, a polyethylene, a polypropylene, a polyester, a polytetrafluoroethylene, a polyamide, a polyimide, and a styrene/isobutylene copolymer.

The device can further include a second layer coated on the first layer, the second layer comprising a polyurethane, a poly(vinyl chloride), a polyamide, an acrylate polymer or copolymer, a polyimide, a polyester, a polycarbonate urethane, an aliphatic urethane, an aromatic urethane, or a cellulose ester. The device can further include a third layer coated on the second layer, the third layer including a polyvinylpyrrolidone, a polyethylene glycol, a polyethylene oxide, or a polyvinylpyrrolidone/vinyl acetate copolymer. The third layer can further include a cellulose ester, a polyamide, an acrylic polymer/copolymer, an epoxy resin, a melamine resin, a formaldehyde resin, a urethane, or a cross-linkable acrylic resin. The cellulose ester can include cellulose nitrate, and the urethane can include aliphatic urethanes, aromatic urethanes and polycarbonate urethanes.

In another aspect, a medical device can have a surface coated with a first layer proximal to the surface, the first layer including a cellulose ester, a urethane, a melamine resin, a formaldehyde resin, and a cross-linkable acrylic resin. The device can include a second layer coated on the first layer, the second layer including one or more of a nitrocellulose, an aliphatic urethane, an aromatic urethane or a polycarbonate urethane.

In another aspect, a method of making a medical device forming a first layer on a surface of the device, the first layer including an ethylene/acrylic acid copolymer. The method can further include forming a second layer on the first layer, the second layer including an aromatic polycarbonate based polyurethane. The method can further include forming a third layer on the second layer, the third layer including a polyvinyl pyrrolidone.

Forming the first layer can include contacting the surface of the device with a solution including the ethylene/acrylic acid copolymer, an epoxy resin, tetrahydrofuran, dimethyl acetamide, anisole, and xylenes, and drying.

Forming the second layer can include contacting the first layer with a solution including an aromatic polycarbonate based polyurethane, a nitrocellulose, dimethylacetamide, anisole, methyl ethyl ketone, and n-butanol, and drying.

Forming the third layer can include contacting the second layer with a solution including a polyvinylpyrrolidone, a nitrocellulose, 4-butyrolactone, ethanol, benzyl alcohol, cyclohexanone, and isopropanol, and drying.

Advantageous properties of the coating modified medical devices allows surgeons to maintain very fine control of surgical procedures, so as to minimize the invasiveness of such surgical procedures, and thereby to enhance patient recovery and surgical outcome.

The details of one or more embodiments are set forth in the description below. Other features, objects and advantages will be apparent from the description, the drawings, and the claims.

### BRIEF DESCRIPTION OF THE DRAWINGS

FIG. 1 shows a perspective view of a marking knife.

FIG. 2 illustrates a partial perspective view of the cutting portion of a marking knife.

FIG. 3 is a depiction of an intravascular retrieval snare.

### DETAILED DESCRIPTION

A medical device can have a coating on an external surface. The coating can be abrasion resistant, lubricious, and biocompatible. A portion of, or the entire surface of, the medical device may be provided with a coating. In some cases, a portion of the device (such as, for example, a handle) is uncoated so that the uncoated portion provides a higher friction surface than a coated portion. In some cases, an inner portion (such as a part or all of a lumen) may be provided with a coating.

Coatings may be continuous or discontinuous (e.g., patterned or covering only portions of the surface) and may be of uniform thickness or may be of uneven thickness. Coatings may be deposited into divets, voids, or grooves in the structure of the device to provide discrete deposits of material. The coating can be applied selectively to a surface of a device, such that desired portions of the surface are coated while other portions remain uncoated. The coating can be discontinuous, i.e., there can be local regions that lack coating, whether the discontinuous nature is desired or unintentional.

The medical device (or at least a portion of it) is be coated with at least one layer, providing the top coat or external layer. The external layer can be directly adhered to a surface of the device. Alternatively, one or more intermediate layers are present between the surface of the device and the external layer.

The medical devices can be coated by applying a coating material to the surface of the medical device. For example, the coating material can be dissolved in a solvent, the resulting solution contacted to the device, and the solvent removed. The coating may be applied using standard coating methods, such as by spraying, dipping, roll coating, bar coating, spin coating, or wiping, or may be manufactured using an extrusion process. The coating may be applied as a solution, and then the solvent allowed to evaporate. Evaporation can be promoted by an elevated temperature. In some cases, kits include a medical device, coated or uncoated and are provided with a swab, which can be wetted with a coating material for coating the surface of the instrument. The kit can be useful in circumstances where it is desirable to apply the coating to the device a short time before the device is used in a medical procedure.

When the coating includes more than one layer (i.e., at least one intermediate layer in addition to the external layer), the layers can be sequentially applied to the device to form the coating. For example, if the coating includes one intermediate layer and an external layer, the intermediate layer can be applied to the device and dried; the external layer is then subsequently applied. The surface of the device can be substantially inert. For example, the surface can be substantially free of reactive functional groups. In some cases, “substantially free of reactive functional groups” simply means that the surface is used as-prepared, with such reactive functional groups as may normally be present in a surface of that particular material, and no exogenous reactive functional groups are added to the surface.

An intermediate layer can be a bonding layer selected to promote adhesion of the external layer to the device. The bonding layer can promote abrasion resistance of the outer layer, and prolong adhesion of the outer layer to the after soaking in water, when compared to a coating without the bond coat layer. The coating can remain adhered to the device when subjected to bending through a small radius. Depending on the substrate material, an additional primer (pre-coat) layer may be used to further improve the adhesion of the bonding and/or lubricious coating layers to the substrate.

Examples of solvents useful for applying the coatings to a device can include butyrolactone, alcohols (e.g., methanol, ethanol, isopropanol, n-butyl alcohol, i-butyl alcohol, t-butyl alcohol, and the like), dimethyl acetamide, and n-methyl-2-pyrrolidone. These solvents and others cause different degrees of swelling of a plastic substrate or inner layer, as the case may be. The duration and temperature of solvent evaporation may be selected to achieve stability of the coating layer and to achieve a bond between the surface being coated and the coating layer. It is possible to control the degree of stability, wet lubricity, insolubility, flexibility, and adhesion of the coating by varying the weight-to-volume percentages of the components in the coating solutions.

The coating can be dried at temperatures between 50° C. and 120° C., but may be done at higher or lower temperatures. Advantageously, hydrophilic coatings can cure faster than a silicone based coating. After drying, the top coat polymer layer is left partially embedded in a polymer surface and/or partially in the case of the two-layer system; the solvent used during the coating application can be too active such that the top coat penetrates into the polymer surface to such a degree that the coated layer behaves as though it has been highly cross-linked. This causes the top coat to become not sufficiently swollen and lubricious when wet by aqueous fluids. Solvent mixtures can also be too inactive, so that the coating is not resistant enough to abrasion when wet and is too easily removed. Other polymers or cross-linking agents may be incorporated with the hydrophilic polymer(s) in the lubricious layer to enhance the adhesion of the layer to the polymer surface, making the lubricious layer more resistant to wet abrasion.

The external layer can include a hydrophilic polymer. The hydrophilic polymer of the external layer can include poly(vinylpyrrolidone) (PVP) or a PVP-vinylacetate copolymer. The hydrophilic polymer of the external layer can have a molecular weight of, for example, greater than 100,000, greater than 150,000, greater than 200,000, greater than 250,000, greater than 300,000, greater than 350,000, or greater than 400,000. In some cases, the hydrophilic polymer of the external layer has a molecular weight in the range of 120,000-360,000. An intermediate layer can include PVP of lower molecular weight, e.g., as low as 15,000. A PVP-vinylacetate copolymer can be used in place of PVP.

The external layer can include a stabilizing polymer. A stabilizing polymer may serve as a component in a coating layer to help bind the lubricious polymer (e.g., PVP) or the coating containing the lubricious polymer, to the device or to the intermediate layer(s) already coated on the device. In this capacity, stabilizing polymers may function as a binding agent that reduces the aqueous solubility of the lubricious polymer, while sustaining the coating's lubricity. Stabilizing polymers also may aid in the co-mingling of the different layers, such that there is molecular mingling at the interface between the two layers. Improved binding of the lubricous layer to the device allows for retention of the coating for longer periods of time and ensures that lubricity is maintained. For example, the stabilizing polymer can be a water-insoluble cellulose polymer (e.g., nitrocellulose), polymethylvinylether/maleic anhydride, acrylic or methacrylic polymers, ethylene acrylic acid copolymers, styrene acrylic copolymers, polyvinyl acetals, ethylene vinyl acetate copolymer, polyvinyl acetate, epoxy resins, phenolic resins, copolymers, or nylon, or a combination thereof. These stabilizing polymers may, optionally, be cross-linked. In some cases, a water-insoluble cellulose polymer is preferable as a stabilizing polymer, for ease of handling and for tendency to produce coatings with greater long-term wet abrasion resistance than coatings prepared with other stabilizing polymers. When the stabilizing polymer is nitrocellulose, a plasticizing agent can be used in conjunction with the nitrocellulose.

For example, the stabilizing polymer can be a water-insoluble cellulose polymer (e.g., nitrocellulose), polymethylvinylether/maleic anhydride, or nylon, or a combination thereof. In some cases, a water-insoluble cellulose polymer is preferable as a stabilizing polymer, for ease of handling and for tendency to produce coatings with greater long-term wet abrasion resistance than coatings prepared with other stabilizing polymers. When the stabilizing polymer is nitrocellulose, a plasticizing agent can be used in conjunction with the nitrocellulose.

The external layer can additionally include materials such as other polymers, plasticizers, reactive agents such as anti-infective materials, colorants such as dyes and pigments, and the like.

An exemplary solution for applying an external layer to a surface can include PVP in a range from 0.01% to 30% w/w of the coating solution polymer component, preferably from 0.5 to 20% w/w, and more preferably 1% to 8% w/w. The amount of stabilizer polymer can range from 0.01% to 20% w/w, preferably from 0.05% to 10% w/w, and more preferably 0.01 to 5% w/w. Preferred commercial sources of the pyrrolidone include International Specialty Products (ISP). Preferred commercial sources of the stabilizer include Hagedorn Akteingesellschaft Chemical. Useful ratios of polyvinylpyrrolidone to stabilizing polymer range from 0.04/99.96 to 99.97/0.03 in the coating solutions.

The solution which forms the external layer can be applied to a deposited coating formed from a mixture of polyurethane or polycarbonate-based polyurethane and stabilizer such as cellulose nitrates.

In a solution used for preparing an intermediate layer, the amount of polyurethane or polycarbonate-based polyurethane can range from 0.05% to 40%, preferably from 0.1% to 20%, and most preferably 3% to 12%. The amount of stabilizer polymer can range from 0.1% to 10%, preferably from 0.5% to 7%, and most preferably 1% to 5%. Polyvinylpyrrolodone is available from BASF and ISP in various molecular weight grades. Preferred commercial sources of the polyurethane and polycarbonate-based polyurethane include Cardiotech International and Thermedics, Inc. Preferred commercial sources of the stabilizer include Hagedorn Akteingesellschaft, I.C.I. and Nobel Enterprises. Cellulose nitrates are available in various viscosity and nitration grades from Hagedorn Akteingesellschaft.

In some cases, a primer layer may be applied directly to a relatively inert medical device/instrument surface that lacks reactive functional groups. Either the solution which forms the external layer or an intermediate layer can be applied to the primer. In a solution for applying a primer layer, the amount of primer polymer may range from 0.5% to 6% w/v, preferably from 1% to 4% w/v, and most preferably 1.5% to 3% w/v. The amount of stabilizer polymer can range from 0% to 10% w/v, preferably from 0.2% to 6% w/v, and most preferably 0.3% to 3% w/v. See, for example, U.S. Pat. No. 6,306,176, which is incorporated by reference in its entirety.

Any of the foregoing coating compositions may contain stabilizers, plasticizers, fillers, and the like ranging from, for example, 0.01% to 70% weight %. In addition, these coating compositions may contain crosslinking agents, in amount ranging from 0.01% to 30% weight %.

The lubricious external layer can be formed on an intermediate layer on a surface of the medical device, the intermediate layer being an adherent, flexible hydrogel coating, e.g., as described in U.S. Pat. Nos. 5,997,517, 6,306,176, and 6,110,483, issued to Whitbourne, et al., each of which is herein incorporated by reference in its entirety. The surface of the medical device may be coated with a coating that includes: (a) a stabilizing polymer such as, for example, optionally crosslinked acrylic or methacrylic polymers, ethylene acrylic acid copolymers, styrene acrylic copolymers, polyvinyl acetals, ethylene vinyl acetate copolymer, polyvinyl acetate, epoxy resins, amino resins, phenolic resins, copolymers thereof, and combinations thereof; and (b) an active agent which can be, for example, a hydrophilic polymer selected to interact with the stabilizing polymer so as to produce a lubricious hydrogel.

The surface of the medical device can be an inert surface, e.g., one that is substantially free of reactive functional groups. The inert surface can be modified by a biocompatible surface coating that includes: (a) an intermediate layer having a thickness below about 100 microns such that the intermediate layer does not penetrate more than superficially into the device. The intermediate layer can include at least one bonding polymer bonded non-covalently with the inert surface of the device, and the intermediate layer can include a cross-linked matrix. The biocompatible surface coating can further include (b) an external layer applied to the intermediate layer that adheres to the intermediate layer, the coating remaining adherent to the surface and resistant to abrasion and to removal from the device after soaking in water relative to a coating without the intermediate layer.

The compositions and/or solutions used to coat the medical devices can be used on mesh, wiry, porous, non-porous, flat and/or sharp surfaces, whether made of metal, ceramic or polymeric substrates or surfaces other that may be used in medical devices.

The coating can have beneficial characteristics for use on the surfaces of devices such as biomedical implants. The coating can be hydrophilic, absorbing water and swelling in an aqueous environment to become a hydrogel. The coating can have lubricant properties, and can be significantly more slippery when wet than when dry. Instruments coated with the described coatings decrease the amount of frictional force required to penetrate tissue. Thus, medical devices coated with the described lubricious compositions are capable of penetrating into or through tissue more easily than a comparable uncoated instrument. The coating can be thin, e.g., on the order of magnitude of one thousandth of an inch. The coating can be coherent and resistant to removal by wet abrasion, and can adhere to a wide variety of substrates. The coating employs biocompatible substances that are neither toxic nor irritating. Surprisingly, the coating may be applied without undesired coating buildup at the cutting edge of the blade, thus minimizing dulling of the blade normally associated with coating of knives and blades. The functional characteristics of the coating may be varied as appropriate for many different applications.

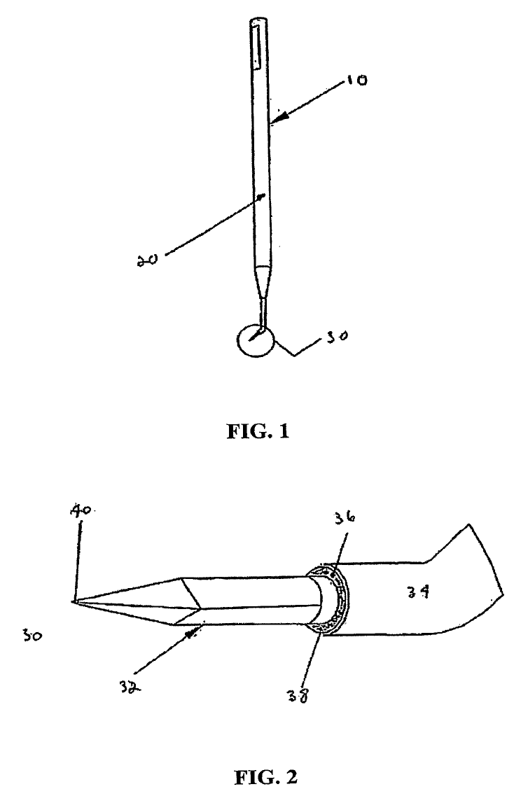

The coating can contain dyes, stains, or pigments or salts useful in diagnosis. The structure of FIGS. 1 and 2 can be coated. See, for example, WO 2005/110302, which is incorporated by reference in its entirety. Referring to FIG. 1, marking knife 10 includes a handle 20 and a cutting portion 30. Referring now to FIG. 2, the cutting portion 30 includes a cutting blade 32 which is held in a retaining means 34, such as a tube, sheath or slot that may be integrally formed with the handle 20 or attached thereto as a separate structure. The blade 32 may be permanently mounted into the retaining means 34 or may be frictionally fit if the remainder of the knife 10 is intended for more than one use. One will also note in FIG. 2 that the retaining means 34 may be formed such that it presents the cutting blade 30 at an angle away from being perpendicular with the handle 20.

Blade 32 may be formed in any one of the conventional shapes known in the art such as a flat blade or a multi-surface blade (as is shown in FIG. 2) and may be curved or rounded. The proximal end of the blade 32 can terminate with a shoulder 36. The shoulder can be relatively flat and perpendicular to the cutting edge of the blade 32. It is contemplated that the shoulder 36 may have a different geometry, such as being convex or concave, depending on the intended use of the surgical knife 10. A bioreactive stain or dye 38 dissolved or disperse in the coating is placed onto the shoulder 36. The stain can be placed on the shoulder 36 during manufacture and is either provided in a dry form dried directly on the shoulder 36 whereby the stain is otherwise stable until hydrated. Such bioreactive stains or dyes include, but are not limited to, Gentian violet, Indocyanine green, Methylene blue, Cresyl blue, VisionBlue and Trypan blue.

The composition of the coating can be varied to control lubricity, swelling, flexibility, and resistance to removal by wet abrasion. These characteristics of the coating can thus be adjusted for various substrates and applications. The solutions used to prepare the invention can have good shelf stability and remain substantially free of precipitate for periods in the range of months or years, so that various mixtures of the solutions for coatings may be prepared at one time and used to coat substrates later. Alternatively, the hydrophilic and stabilizing polymers, and if desired, a plasticizing agent and an adherent polymer, may even be prepared in a single solution. Furthermore, because the use of chlorinated solvents or other acute toxics is not required, fewer precautions are necessary to protect workers from health hazards.

The stabilizing polymers, particularly modified cellulose polymers, can be able to make hydrophilic polymers, such as PVP and PVP-vinyl acetate copolymers, stable and insoluble in water and lower alcohols. The resulting combination, when applied to a substrate, produces a coating that is a stable layer or layers that are bonded to a substrate surface, that is not slippery when dry but is desirably lubricious when wet, and is resistant to removal by conditions of wet abrasion. The coating layer bonds to an impervious surface such as stainless steel or glass. It also bonds to polymer surfaces where the surface interacts with the components of the coating.

Preferably, the solution for depositing the external layer includes a solvent that is to capable of solubilizing (at least partially) components of the external layer and an intermediate layer. As such, the solvent can promote penetration of the external layer components into the intermediate layer, and is believed to bring about a mixing of the components of both layers.

Such mixing can facilitate chemical reactions such as cross-linking between the components, or facilitate physical mixing of layers without chemical reactions. In some embodiments, there can be a high degree of cross-linking or intermolecular mingling between a hydrophilic polymer and a stabilizing polymer at the interface between the external and intermediate layers of the coating. Thus, a region between the two layers may be created as a result of cross-linking or intermolecular mingling between the polymers contained in the separate layers. The slight degree of cross-linking or mingling at the outer surface of the coating can aid in providing the lubricity of the coating.

In practice, the composition of the solvent mixture can be adjusted so that the degree of penetration of the external layer into the intermediate layer is in a useful range. For example, if the external layer solvent mixture is too active toward the intermediate layer, then too much penetration into the intermediate layer occurs, and the external layer may be rendered less lubricious when wet than desired. Conversely, if the external layer solvent is too inactive toward the intermediate layer, then too little penetration of the external layer into the intermediate layer occurs, and the coating may be too easily removed from the inner layer by wet abrasion.

An exemplary coating can be applied to the surface of a medical device with sufficient thickness and permanence to retain the coating's desirable qualities throughout the useful life of the coated device. The coatings are desirably non-reactive with living tissue and may be non-thrombogenic in blood.

When tested by subjective methods, the coatings when wet, are more slippery than wet, greased glass, and, when dry, are no more slippery than dry glass. The coatings are resistant to removal by wet abrasion as determined by running water over the coatings and rubbing between tightly gripped fingers while wet. The coatings have high adherence when dry, as determined by attaching adhesive tape, pulling the tape off with a vigorous action, and then wetting the coated substrate to determine whether the taped portion retained the lubricant coating. The coatings remain adherent and coherent for extended periods when stored in water, and neither peel off, dissolve, nor dissociate.

Other approaches to produce adherent coatings on difficult-to-coat substrates such as metals (e.g., stainless steel or titanium) can include using a primer layer.

Adherent coatings may be prepared using plasma treatment prior to coating. Plasma treatment (e.g., oxygen or nitrogen plasma) may also be used to introduce functional groups on the surface which may further improve adhesion of the coating to the device.

In an exemplary embodiment, a coated medical device, e.g., a knife or scalpel, is removed from its sterile wrapper, is gripped by a user (e.g., surgeon), the blade is dipped in water to make the blade slippery, and the blade is used to make an incision while the blade is wet. In another exemplary embodiment, a coated medical device, such as an intravascular retrieval snare system, is removed from its sterile wrapper, is gripped by a user (e.g., surgeon), the snare is dipped in water to make the coating slippery, and the snare is placed into a vein or artery and monitored by ultrasound imaging. The snare is then used to retrieve foreign objects from the patient's vascular system. In other embodiments, the blade or snare is provided in a wetted state, and does not need to be wetted by a user before use.

In some embodiments, tools or devices useful in surgical procedures, such as dissection, curetting, suturing, and cutting with scissors, a surgical knife, blade, or a scalpel, are provided with a lubricious and optionally echogenic coating. The surgical instruments may include rongeurs, dissectors, knives, scalpels, scissors, needle drivers, suture holders, curettes, electrodes, probes, forceps, aneurysm clip applicators, and the like. The surgical instruments can be scalpels and knives (e.g., opthalmology knives). For example, the surgical knife may be a SHARPOINT ophthalmic blade.

The surgical instruments can be catheters, guide wires, or medical tools. Catheters include, for example, PTCA catheters, cardiology catheters, central venous catheters, urinary catheters, drain catheters, and dialysis catheters. The catheter may be a percutaneous biliary drainage catheter which may come in different lengths for the biliary procedure, one of which reaches the duodenum for correct placement of a guide wire, and may further include a locking pigtail. Other exemplary catheters include a nephrostomy catheter, of e.g. soft polyurethane for optimal kink resistance, with or without plastic and metal stiffener, with or without large drainage holes to provide minimal tissue trauma and ulcerations, and thereby reduce patient discomfort. The surgical instruments can be connecting tubes for drainage bags. Guidewires, as well as tips of wire guides used in conjunction with, e.g., catheters, can be coated.

The surgical instruments can be introducer sets, which for catheters include a co-axial system for placement of a guide wire in non-vascular procedures. The system includes a coaxial dilator (which can be coated or uncoated) and at least one guide wire, which can be coated or uncoated. Guidewires include CANALIZER wires from InterV/PBN Medicals Inc.

Other catheter/tube medical devices which can be prepared with the coatings include: abdominal cavity drainage, ablation catheters, angiography catheters, angioplasty balloons, arterial line, artificial insemination catheters, Bivona tracheostomy tubes, catheters, cavity drainage catheters, central venous catheters, cholangiography, cutting loops, diagnostic electrode catheters, dilation balloons, drainage catheters, electrode catheters, embolectomy catheters, endobronchial tubes, endotracheal tubes, epidural catheters, Foley catheters, guiding catheters, hemodialysis catheters, Kumpe access catheters, laryngectomy tubes, laser ureteral catheters, pacing catheter, percutaneous access/catheter sets, percutaneous enteral feeding devices, peripheral catheters, PICC lines catheters, pigtail ureteral catheters, rectal pressure catheters, renal access catheter, stimulating catheter, suction catheters, thermodilution intra-aortic balloon catheters, tracheostomy tubes, ureteral drainage, urinary catheters, urodynamics catheters, and wedge pressure catheters.

Surgical needles and sutures can be coated. Sutures and suture needles (which may be attached to the suture) may be coated with a lubricious and/or echogenic coating as described herein. The entire needle or suture or only a portion of the needle or suture may be coated. It may be practical to coat just the distal ⅔ of a suture needle leaving the proximal end uncoated, such that it is more easy to grip by the surgeon during use. Among the needles that can be coated are needles of between ⅛ and ½ circle with dimensions of between 1 and 100 mm, for example, between 5 and 50 mm. The needle can be, for example, a side cutting lancet needle; a reverse cutting needle; a precision reverse cutting needle; an ULTRAGLIDE needle; or a DERMAGLIDE needle. More particularly, coated needles can be of ⅛ circle having dimensions of 5.51 and 14.99 mm; needles of ⅜ circle having dimensions of 6.15, 6.6, 6.15, 6.68, 11, 12, 13, 14, 16, 18, 24, 30, 36 or 40 mm; ¼ circle needles with dimensions of 6.6, 8, or 8.51 mm; needles of ½ circles with dimensions of 9, 16, 15, 16, 18, 20, 24, 26, 27, 37, or 40 mm; or bicurve needles of 4.8 or 5.51 mm. Other needles used in medical application which can be produced include: amniocentesis needles, brachytherapy needles, core tissue biopsy needles, docking needle discograms, epidural needles, facial incision needles, needles for delivering anesthesia, such as peritubular and retrobulbar needles, Huber needles, insulin pump needles, lumbar puncture needles, nerve block needles, procedural needles, prostate biopsy needles, or vertebroplasty needles.

Cutting instruments, such as scalpels, knives and scissors, whether disposable or not, and other cutting devices may be coated. Suitable coated cutting instruments include micro-blade; slit knives (in which the blade is optionally angled) with a knife size of 2.5 to 5.0 mm; stab knives; incisional instruments; sideport knives; capsulotomy instruments; surgical blades including carbon steel blades; stainless steel blades; surgical knives and microsurgical knives; scalpels; safety scalpels blade remover; scalpel cartridges; shave biopsy devices; or safety prep razors. In an exemplary embodiment, the surgical instrument may be a precision knife for micro-incisions. The blade of the instrument may comprise about 1.3 mm to about 1.6 mm in width. The dimensions of the blade may be 1.5 mm×1.7 mm, 1.5 mm×2.0 mm or 1.7 mm×2.0 mm. In some embodiments, the blade may be in sizes of about 0.6 mm, 0.8 mm, or 1.1 mm. Other cutting instruments that can be coated include: dissection knives, electrosurgical bipolar, corneal blades, or corneal punches. The blade of a surgical instrument may be flat, have different geometrical shapes and/or featuring tapered facets.

In some embodiments, the entire surface of the blade may be coated with one or more of the described coatings. Alternatively, the coating is applied to only the tip or blade of the surgical instrument. There may be a number of different configurations of working tips or blades such as round knife blades, probes, blunt blades, curved blades, suture holders, needle holders, scissors, curettes and the like. The coating may be applied to only the cutting edge of the instrument.

In another exemplary embodiment the coating is applied to the tip of the surgical instrument only or to the whole snare including the catheter portion. Other instruments providing a substrate treatment in accordance with the invention include condoms, contact lenses, peristaltic pump chambers, arteriovenous shunts, gastroenteric feed tubes and endotracheal tubes, or other implants of metal or polymer substrate.

Other medical equipment which can be produced include: angiography, balloon stents, bone biopsy device, cement delivery system, electrosurgical electrodes, embryo replacement, guidewires, hemodialysis products, joint anchors interference, screws & fixation, neuro micro-driver, osteo introducer, percutaneous sheath introducers, peripheral nerve block (PNB), punctum plugs, RF thermoablation, Roadrunner PC wire guides, spinal fixation, spinal implants, spinal screws, stone baskets, thoracentesis, ureteral stents, urethral dilation, uretral stents, wire guides, bone cement delivery device, bone filler device, cortex extractors, cannulae (e.g., irrigation and aspiration cannulae), lens loops, stromal puncture needles, bone tamp, cystotome, pacing electrodes, paracentesis, stone manipulating devices, two part trocar sets, braided sutures, or endoscopic suturing devices.

Sutures

The lubricious coating can be applied to a surface of a suture, for example a silk microsuture, e.g., one having a diameter of less than 0.5 mm, 0.4 mm or less, 0.3 mm or less, or 0.2 mm or less. The sutures can be monofilamentary or multifilamentary, and can be plain or self-retaining, the self-retaining sutures having retainers (such as barbs or other tissue gripping structures) that engage when the suture is pulled in a direction other than that in which it was deployed in the tissue. A self-retaining suture may be unidirectional, having retainers oriented in one direction along the suture body, or bidirectional, having one of more retainers near one of the suture oriented in one direction and one or more retainers near the other end of the suture oriented in the opposite direction. Retainers may be configured to have tissue insertion points (such as barbs), tissue insertion edges (such as conical or frusto-conical structures), etc., and can help to anchor the sutures in place once introduced by a surgeon. The suture can be introduced so that the retainers do not engage while the suture is being placed. With a self-retaining suture, the coating can be only on an exterior surface of a retainer. For example, the coating can be applied to the suture before the retainers are formed, so that when the retainers engage, the engaging surface is substantially free of the coating. In this way, tissue being sutured contacts a lubricious surface of the suture as the suture is introduced, but when the retainer engages, a non-coated surface of the retainer contacts the tissue. The non-coated surface of the retainer can provide greater friction to the tissue than the coated surfaces of the suture, providing additional anchoring.

The lubricious coating may be applied to various types of sutures. The sutures may be absorbable (e.g., those that are degraded by the body's enzymatic pathways and generally lose tensile strength by 60 days after implantation), and may be made of polymers or copolymers of glycolic and lactic acid. Exemplary absorbable sutures include catgut (both plain and chromic) (e.g., those with a trade name PROGUT from Dolphin Sutures, India), and those derived from polyglycolic acid with a trade name PETCRYL (Dolphin Sutures, India) and with a trade name DEXON™ (Sherwood Services AG, Schaffhausen, Switzerland), from poliglecaprone 25 with a trade name MONOCRYL® (copolymer of about 75% glycolide and about 25% caprolactone, Johnson & Johnson Co., New Brunswick, N.J.), from polyglactin 910 (such as VICRYL®, coated VICRYL®, coated VICRYL® Plus Antibacterial sutures that contain antibacterial triclosan, and Coated VICRYL RAPIDE® sutures, Johnson & Johnson Co., New Brunswick, N.J.), MULTIPASS® Needle Coating (Johnson & Johnson Co., New Brunswick, N.J.), copolymer of about 67% glycolide and about 33% trimethylene carbonate sold as MAXON™, Wyeth, Madison, N.J., and from polydioxanone with a trade name PDS II® (Johnson & Johnson Co., New Brunswick, N.J.).

In addition to the sutures described above, degradable sutures can be made from polymers such as polyglycolic acid, copolymers of glycolide and lactide, copolymers of trimethylene carbonate and glycolide with diethylene glycol (e.g., MAXON™, Tyco Healthcare Group), terpolymer composed of glycolide, trimethylene carbonate, and dioxanone (e.g., BIOSYN™ glycolide (60%), trimethylene carbonate (26%), and dioxanone (14%), Tyco Healthcare Group), copolymers of glycolide, caprolactone, trimethylene carbonate, and lactide (e.g., CAPROSYN™, Tyco Healthcare Group). These sutures can be in either a braided multifilament form or a monofilament form. The polymers can be linear polymers, branched polymers or multi-axial polymers. Examples of multi-axial polymers used in sutures are described in U.S. Patent Application Publication Nos. 20020161168, 20040024169, and 20040116620, each of which is incorporated by reference in its entirety.

Absorbable sutures may be used below the surface of the skin to provide support to the skin closure. They may also be used in areas where suture removal might jeopardize the repair such as with small children who might not easily cooperate with suture removal.

Sutures on which the lubricious coating may be applied may also be non-absorbable. Non-absorbable sutures are permanent and include sutures made of polyamide (also known as nylon, such as nylon 6 and nylon 6.6), polyester (e.g., polyethylene terephthlate), polytetrafluoroethylene (e.g., expanded polytetrafluoroethylene), polyether-ester such as polybutester (block copolymer of butylene terephthalate and polytetra methylene ether glycol), polyurethane, metal alloys, metal (e.g., stainless steel wire), polypropylene, polyethelene, silk, and cotton. Exemplary non-absorbable sutures include coated polyester sutures with a trade name Procare (Dolphin Sutures, India), GORTEX™ (made of expanded polytetrafluoroethylene, sold by Gore), NOVAFIL™ (made of polybutester, Wyeth, Madison, N.J.), monofilament polyamide sutures with a trade name Linex (Dolphin Sutures, India), SUTURA® (black braided silk sutures, Sutura Inc., Fountain Valley, Calif.), monofilament polypropylene sutures with a trade name Duracare (Dolphin Sutures, India), MONOSOF® (monofilament nylon suture, United States Surgical Co., Norwalk, Conn.), DERMALON™ (monofilament nylon suture, Sherwood Services AG, Switzerland), SURGILON™ (braided nylon suture coated with silicone, Sherwood Services AG, Switzerland), Ethilon nylon suture (Ethicon, Inc., Somerville, N.J.), ETHIBOND EXCEL® (braided polyester suture from Johnson & Johnson Co., New Brunswick, N.J.), Pronova poly(hexafluoropropylene-VDF) suture (Ethicon, Inc. Somerville, N.J.), TEVDEK™ (braided polyester suture from J.A. Deknatel and Son, Inc. New York, N.Y.), PROLENE™ (polypropylene suture from Ethicon, Inc., Somerville, N.J.), FLUOROFIL™ (polypropylene suture from Pitman-Moore, Inc. Lake Forest, Ill.), and MERSILENE™ (polyester fiber suture from Ethicon, Inc., Somerville, N.J.).

Additional exemplary sutures to which the lubricious coating may be applied are various sutures available from Surgical Specialties Co., Reading, Pa.), including monoderm undyed or dyed monofilament sutures, clear or dyed PCL monofilament sutures, dyed polypropylene monofilament sutures, undyed braided POLYSYN FA sutures, dyed or undyed braided PGA sutures, dyed or undyed braided polysyn suture, dyed monofilament polysyn sutures, dyed braided polyester sutures, braided silk sutures, dyed braided polyviolene sutures, plain or chromic gut sutures, dyed or undyed monofilament nylon sutures, or dyed pliable nylon sutures.

Additional exemplary sutures to which the lubricious coating may be applied are various sutures available from Tyco International Ltd., Bermuda or its companies. Such sutures include SURGITIE™ (single use ligating loops with delivery system) and SURGIWIP™ (single use suture ligatures with delivery system), absorbable sutures such as POLYSORB™ (sutures composed of LACTOMER™ glycolide/lactide copolymer, a synthetic polyester composed of glycolide and lactide (derived from glycolic and lactic acids), DEXON™ II (synthetic suture composed of homopolymer of glycolic acid and coated with POLYCAPROLATE™, a copolymer of glycolide and epsilon-caprolactone), DEXON™ S (synthetic sutures composed of the homopolymer of glycolic acid), MAXON™ CV (polyglyconate synthetic sutures prepared from a copolymer of glycolic acid and trimethylene carbonate), plain, mild chromic, and chromic gut sutures composed of purified connective tissue (mostly collagen) derived from the serosal layer of beef intestines, and non-absorbable sutures such as DERMALON® (nylon), MONOSOF® (nylon), SURGILON® (nylon), SURGIDAC™ (polyethylene terephthalate), TI-CRON™ (sutures prepared from fibers of high molecular weight, long chain and linear polyesters having recurrent aromatic rings as an integral component), SURGIPRO™ (sutures composed of an isotactic crystalline stereoisomer of polypropylene (a synthetic linear polyolefin) and polyethylene), SURGIPRO™ II (sutures composed of an isotactic crystalline stereoisomer of polypropylene (a synthetic linear polyolefin) and polyethylene), NOVAFIL™ (sutures composed of polybutester, a copolymer of butylenes terephthalate and polytetramethylene ether glycol), VASCUFIL™ (sutures composed of a copolymer of butylenes terephthalate and polytetramethylene ether glycol and coated with POLYTRIBOLATE™, an absorbable polymer of ε-caprolactone/glycolide/poloxamer 188), FLEXON™ (twisted multistrand steel sutures coated with orange or white PTFE poly(tetrafloroproethylene) or clear FEP poly(tetrafluoroethylene-co-hexafluoropropylene), SOFSILK™ (sutures composed of natural proteinaceous silk fibers that are treated to remove the naturally-occurring sericin gum), and stainless steel sutures.

In certain embodiments, sutures to which the lubricious coating may be applied are used for joining tissue in surgical procedures including, without limitation, joining and holding closed a wound (such as a surgical incision) in bodily tissue, fastening junctions of wounds, tying off wounds, and joining a foreign element to tissue.

In certain embodiments, sutures to which the lubricious coating may be applied are used in various dental procedures, i.e., oral and maxillofacial surgical procedures, and thus may be referred to as “dental sutures.” The above-mentioned procedures include, but are not limited to, oral surgery (e.g., removal of impacted or broken teeth), surgery to provide bone augmentation, surgery to repair dentofacial deformities, repair following trauma (e.g., facial bone fractures and injuries), surgical treatment of odontogenic and non-odontogenic tumors, reconstructive surgeries, repair of cleft lip or cleft palate, congenital craniofacial deformities, and esthetic facial surgery. Many of the various sutures described above are used in such procedures and are available from many of the same commercial sources. As above, dental sutures may be degradable or non-degradable. Sutures used in oral and maxillofacial surgical procedures may typically range in size from USP 2-0 to USP 6-0. Dental sutures may have a surgical needle attached.

In certain embodiments, self-retaining sutures to which the lubricious coating may be applied are used in tissue repositioning surgical procedures. Such surgical procedures include, without limitation, face lifts, neck lifts, brow lifts, thigh lifts, and breast lifts. Self-retaining sutures used in tissue repositioning procedures may vary depending on the tissue being repositioned; for example, sutures with larger and further spaced-apart retainers may be suitably employed with relatively soft tissues such as fatty tissues.

In certain embodiments, sutures to which the lubricious coating may be applied are microsutures. Microsutures are used in microsurgical procedures that are performed under a surgical microscope. Such surgical procedures include, but are not limited to, reattachment and repair of peripheral nerves, spinal microsurgery, microsurgery of the hand, various plastic microsurgical procedures (e.g., facial reconstruction), microsurgery of the male or female reproductive systems, and various types of reconstructive microsurgery. Microsurgical reconstruction is used for complex reconstructive surgery problems when other options such as primary closure, healing by secondary intention, skin grafting, local flap transfer, and distant flap transfer are not adequate. Microsutures are available from many of the commercial sources identified above and are made from the same materials described above. As above, microsutures may be degradable or non degradable. Microsutures have a very small caliber, often as small as USP 9-0 or USP 10-0, and may have an attached needle of corresponding size.

Additional exemplary sutures to which the lubricious coating may be applied are described in U.S. Pat. Nos. 5,766,188, 4,441,496, 6,692,516, 4,550,730, 4,052,988, and U.S. Patent Application Publication Nos. 2005267532, 2005240224, 2004111116, 2004088003, 2002095180, each of which is incorporated by reference in its entirety.

Sutures to which the lubricious coating may be applied may be commercially available or may be made using any suitable method, including injection molding, stamping, cutting, laser, extrusion, separate manufacture and subsequent attachment of retainers, and the like. With respect to cutting, polymeric thread or filaments may be purchased, and retainers subsequently cut or added onto the filament body. In certain embodiments, barbed sutures may be produced according to U.S. Pat. No. 6,848,152 and U.S. Patent Application Publication Nos. US 200410226427 and US 2004/0060409, each of which is incorporated by reference in its entirety.

In certain embodiments, sutures to which the lubricious coating may be applied are already attached to surgical needles. Attachment of sutures and surgical needles is described in U.S. Pat. Nos. 3,981,307, 5,084,063, 5,102,418, 5,123,911, 5,500,991, 5,722,991, 6,012,216, and 6,163,948, and U.S. Patent Application Publication No. US 2004/0088003. A method for the manufacture of surgical needles is described in U.S. Pat. No. 5,533,982, and a method for the manufacture of polymer-coated surgical needles is described in U.S. Pat. No. 5,258,013, each of which is incorporated by reference in its entirety.

In certain embodiments, the sutures to which the lubricious coating may be applied are pointing at both ends (including suture connectors as described in U.S. Pat. No. 6,241,747, which is incorporated by reference in its entirety). In certain other embodiments, the sutures may have one pointing end and an anchor on the other end. The anchor may be used to secure the implantation of the suture in soft tissue (e.g., those described in U.S. Patent Application Publication No. US2005/0267531, which is incorporated by reference in its entirety) or the attachment of sutures to the bone (e.g., those described in U.S. Pat. No. 6,773,450 and PCT Application Publication No. WO 2004/014236, each of which is incorporated by reference in its entirety).

In certain other embodiments, the suture may be a relatively short suture with sharp pointing ends. Such a suture may function similar to a staple when used in connecting tissues and thus permits a surgeon to rapidly and securely attach the edges of a wound in a bodily tissue or reconfigure the tissue without the necessity for threading and tying numerous individual stitches or for the use of a complicated tool to insert the suture. This type of sutures may thus be referred to as “suture connector.” In certain embodiments, the suture connector may be a bi-directional self-retaining suture. In certain other embodiments, the suture connector may be found by linking two relatively short uni-directional self-retaining sutures together to form a bi-directional self-retaining suture (see, U.S. Pat. No. 6,241,747, which is incorporated by reference in its entirety).

Catheters

The lubricious coating can be applied to a surface of a catheter or a catheter accessory, such as, for example, a catheter patency device, a centesis catheter, a drainage catheter, one or more components of a guidewire introduction system, one or more components of a hystero-access catheter set, or a vessel sizing catheter.

A centesis catheter can have a variety of dimensions, e.g., from 4 F to SF×7 cm to 20 cm. In particular, a centesis catheter can have dimensions of 4 F×7 cm, 4 F×10 cm, 4 F×15 cm, 5 F×7 cm, 5 F×10 cm, 5 F×15 cm, or 5 F×20 cm. The centesis catheter can have four distal side holes to provide drainage in small cavities, and can have a Luer lock hub for secure, one-handed placement. Exemplary centesis catheters include SKATER® centesis catheters and from InterV.

The drainage catheter can have a variety of dimensions, e.g., from 6 F to 16 F×20 cm to 25 cm. In particular, a drainage catheter can be a 6 F×20 cm locking pigtail catheter that accepts a 0.035″ guide wire, a 7 F×20 cm locking pigtail catheter that accepts a 0.035″ guide wire, a 8 F×25 cm locking pigtail catheter that accepts a 0.038″ guide wire, a 10 F×25 cm locking pigtail catheter that accepts a 0.038″ guide wire, a 12 F×25 cm locking pigtail catheter that accepts a 0.038″ guide wire, a 14 F×25 cm locking pigtail catheter that accepts a 0.038″ guide wire, a 16 F×25 cm locking pigtail catheter that accepts a 0.038″ guide wire, a 6 F×20 cm non-locking pigtail catheter that accepts a 0.035″ guide wire, or a 7 F×20 cm non-locking pigtail catheter that accepts a 0.035″ guide wire. The catheter can be used with Seldinger or Trocar insertion techniques. Exemplary drainage catheters include SKATER® single step catheters and SKATER® drainage catheters from InterV.

A drainage tubing portion of the catheter can be made of a thermoplastic polyurethane elastomer such as pellethane. This portion can be spray-coated with a solution and dried to coat the drainage tubing portion of the catheter with a lubricious coating. In one embodiment, the solution used for spray coating has the composition shown in the following table.

| Component | Weight % |
|---|---|
| Denatured Anhydrous Ethanol (EtOH) | 10.10 |
| Benzyl Alcohol | 18.10 |
| Cyclohexanone | 47.16 |
| Tetrahydrofuran (THF) | 22.40 |
| Nitrocellulose stock solution | 0.14 |
| Polyvinylpyrrolidone K-90 (PVP K-90) | 2.10 |

Nitrocellulose Stock Solution

| | |
|---|---|
| ¼ RS (H27) Nitrocellulose (Manufacturer: Hagedorn) | 9.00 |
| 4-butyrolactone (BLO) | 91.00 |

Coated drainage catheters can be sterilized by treatment with ethylene oxide.

A hystero-access catheter set can include several components for use together during a medical procedure, e.g., selective salpingography or fallopian tube procedures. For example, the set can include a 10 F hystero-access balloon catheter (which can have a non-latex balloon for sealing the cervix), and a 5 F or 7 F selective salpingography catheter, which can accept a 0.035″ guidewire or 3 F catheter for coaxial introduction. The set can include a 0.035″ guidewire. One or more of the components of the hystero-access catheter set can have a surface coated with a lubricious coating.

A vessel sizing catheter can be, for example, a 5 F×90 cm pigtail catheter that accepts a 0.035″ guide wire and has 6 side holes; or a 5 F×65 cm straight catheter that accepts a 0.035″ guide wire and has 10 side holes. Either can include radiopaque bands (e.g., gold bands) at regular intervals for measurement, e.g., during angioplasty. Exemplary vessel sizing catheters include GOLDEN-RULE® vessel sizing catheters from InterV.

Guidewires

A guidewire can be coated with a lubricious coating. The guidewire can be a stainless steel guidewire (e.g., an 0.018″×80 cm stainless steel guidewire with platinum tip), or a nitinol guidewire (e.g., an 0.0188″×40 cm or 0.018″×80 cm nitinol guidewire with platinum tip). Guidewires may be straight or include a curved or coiled portion and may range in flexibility. The guidewire can also include a polyurethane sleeve (e.g., a sheath over a nitinol core). The radiopaque polyurethane sleeve may be radiopaque to promote fluoroscopic visualization. The guidewire can have a straight or pre-angled tip. In some embodiments, the guidewires have a diameter of 0.035″ and a length in the range of 150 cm to 260 cm.

Needles

A variety of needles and accessories can advantageously include a surface having a lubricious coating. For example, the needle can be a biopsy site marker, a bone marrow biopsy needle, a breast localization needle, a component of a galactography kit, an injection needle, a soft tissue biopsy coaxial introducer needle, or a soft tissue biopsy disposable needle (optionally for use with reusable automatic instruments or semi-automatic instruments).

A biopsy site marker can include both a bio-absorbable plug and a permanent anchor, to permanently mark a breast biopsy site, allowing for future identification of the biopsied area. The highly visible plug portion can be absorbed into the breast tissue, while the anchor remains in place allowing for long-term stability and permanent visualization. The bio-absorbable plug provides ultrasound, MRI, and mammography visibility for up to six weeks, and allows for rapid and accurate marker placement under ultrasound guidance. An exemplary biopsy site marker is the V-MARK® sold by InterV.

A bone marrow biopsy needle can have a variety of dimensions, such as, for example, 15 G×2.688″, 15 G×4″, 16 G×2.688″, 16 G×4″, 8 G×4″, 8 G×6″, 11 G×4″, 10 G×6″, 13 G×2″, 13 G×3″, in either I-needle or J-needle form.

A breast localization needle can have a variety of dimensions, such as 20 G×3 cm, 20 G×5 cm, 20 G×7.5 cm, 20 G×10 cm, or 20 G×12.5 cm. The needle can have optionally have a J-shape, and optionally include centimeter markings for depth measurement. The needle can include a side barb and may be flexible or rigid.

A galactography kit can include an injection cannula (e.g., a 24 G or 30 G curved injection cannula) with dilator (e.g., a 0.010 or 0.012 diameter dilator).

An injection needle can be, for example, a multi-pronged injection needle, which can have multiples tines (each with one or more through-holes) for fluid delivery. The needle can have a trocar-style tip. The needle can be an 18 G needle with a length of, for example, 10 cm, 15 cm or 20 cm. The injection needle can be a QUADRA-FUSE needle sold by InterV.

A soft tissue biopsy coaxial introducer needle can be used with a biopsy instrument and optionally with a biopsy site marker. For example, the introducer needle to (e.g., for use with a BioPince® biopsy instrument) can have dimensions of 15 G×6.8 cm, 15 GA×11.8 cm, 17 GA×6.8 cm, 17 GA×11.8 cm, or 17 GA×16.8 cm. The needle can have centimeter markings, and an echogenic tip. Introducer needles for use with SUPERCORE instruments can have dimensions of 13 G×3.9 cm, 13 G×9.9 cm, 15 G×3.9 cm, 15 G×9.9 cm, 17 G×3.9 cm, 17 G×9.9 cm, 17 G×14.9 cm, 19 G×4.2 cm, or 19 G×10.2 cm. Introducer needles for use with TRUCORE instruments can have dimensions of 13 G×5.1 cm, 13 G×11.1 cm, 15 G×5.1 cm, 15 G×11.1 cm, 17 G×5.1 cm, 17 G×11.1 cm, 17 G×15.1 cm, 19 G×5.4 cm, or 19 G×11.4 cm. Introducer needles for use with TRUCORE instruments can have dimensions of 13 G×4.6 cm, 13 G×10.6 cm, 15 G×4.6 cm, 15 G×10.6 cm, 17 G×4.6 cm, 17 G×10.6 cm, 17 G×14.6 cm, 19 G×4.9 cm, or 19 G×10.9 cm. Introducer needles can also be used with PRO-MAG, OSTY-CORE, and ACN biopsy needles.

A soft tissue biopsy disposable needle can be, for example, a MAXICELL needle which can harvest tissue on both forward and backward thrusts of the needle. The needle can have a 30° matched ground needle tip geometry that helps harvest a cluster of intact cells. The needle can have numbered centimeter marks for depth placement. The needle can have an echogenic tip. The needle can have dimensions of 22 G×5 cm, 22 G×9 cm, or 22 G×15 cm. The soft tissue biopsy disposable needle can be used with an introducer needle.

A soft tissue biopsy disposable needle can be a Chiba style, a spinal style, a Franseen style, Westcott style, or a Greene style needle. The needle can have numbered centimeter marks for depth placement. The needle can have an echogenic tip. The needle can have dimensions of, for example, 18 G, 20 G or 220, and a length of 9 cm to 20 cm.

A soft tissue biopsy disposable needle can be a TECHNA-CUT needle, which can have a trocar style needle tip that allows for easy direct puncture while minimizing tissue damage, and a precise cutting edge on the outer cannula that contributes to a complete, intact core specimen. A TECHNA-CUT needle can have dimensions of, for example, 16 G to 23 G and a length of 6 cm to 15 cm.

PRO-MAG needles (e.g., for use with PRO-MAG biopsy instruments) can include a 19 mm sample notch ensures sufficient tissue for clinical diagnosis. The needle can have numbered centimeter marks for depth placement. The needle can have an echogenic tip. The needle can be used with a coaxial introducer needle. A PRO-MAG needle can have dimensions of, for example, 14 G to 20 G, and a length of 10 cm to 30 cm.

A SUPERCORE needle can include an adjustable specimen notch (exposing either 19 mm or 9.5 mm), to provide clinical flexibility. The needle can have numbered centimeter marks for depth placement. The needle can have an echogenic tip. The needle can be used with a coaxial introducer needle. A SUPERCORE needle can have dimensions of, for example, 14 G to 20 G, and a length of 9 cm to 20 cm.

A disposable soft tissue biopsy needle for use with an automatic instrument can be, for example, a needle for use with a BIO-PINCE instrument, which can include a tri-axial cut and trap cannula system, to cut the specimen and hold it in the cannula. Needles for use with the BIO-PINCE instrument can be 16 G or 18 G with a length of 10 cm to 20 cm.

Vascular Interventional Devices

Tools or devices used during medical procedures for foreign body retrieval and manipulation procedures can include a surface with a lubricious coating. Such tools or devices include those as used in the cardiovascular system or hollow viscus to retrieve or manipulate foreign objects. The medical devices can be intravascular retrieval snares or baskets and other laprascopic devices.

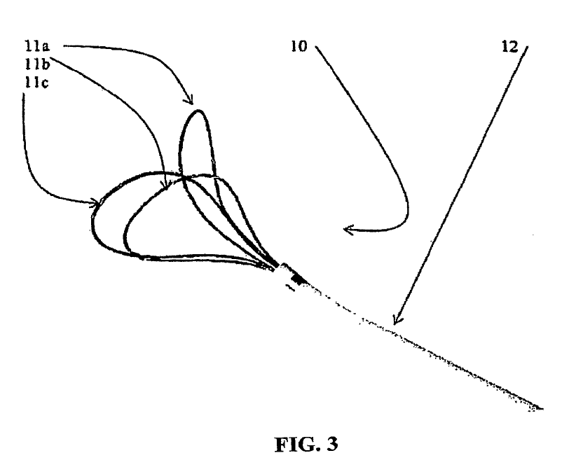

For example, the intravascular retrieval snare can be the EN SNARE system. With reference to FIG. 3, the intravascular retrieval snare can include an intravascular retrieval snare system (10), having a tip including 3 interlaced loops (11a, 11b and 11c) that enables the capture, retrieval or manipulation of objects in a vascular body. The instrument can be rotated during use to provide positive engagement with targeted objects. The tip of the device may comprise of a variety of materials including metals, such as stainless steel or interwoven platinum strands. In another aspect, the tip loops may include super-elastic nitinol wire. The snare system may include a guiding catheter (12) and/or an introducer/back loading device and/or a steering handle. In particular, an outer surface of guiding catheter 12 can be coated with a lubricious coating.

The instrument can be a mini snare with a diameter of from about 1 mm to about 50 mm and a length of from about 100 cm to about 200 cm; and about 3 F to about 7 F by about 120 cm to about 150 cm catheter. For example, the instrument may be a mini snare with 2-4 mm diameter×175 cm length and 3 F×150 cm catheter, a mini snare with 4-8 mm diameter×175 cm length and 3 F×150 cm catheter, a standard snare with 6-10 mm diameter×120 cm length and 6 F×100 cm catheter, a standard snare with 9-15 mm diameter×120 cm length and 6 f×100 cm catheter, a standard snare with 12-20 mm diameter×120 cm length and 6 F×100 cm catheter, a standard snare with 18-30 mm diameter×120 cm length and 7 F×100 cm catheter, or a standard snare with 27-45 mm diameter×120 cm length and 7 F×100 cm catheter.

Other intravascular snare devices, which have a variety of sizes and configurations, may be used in conjunction with the coating. Examples of snares are described in U.S. Pat. No. 6,913,612 to Palmer, et al., U.S. Pat. No. 3,828,790 to Curtiss et al., U.S. Pat. No. 5,171,233 to Amplatz et al., U.S. Pat. No. 5,098,440 to Hillstead and U.S. Pat. No. 6,099,534 to Bates, which are herein incorporated by reference in their entirety.

Other vascular interventional devices include a non-invasive or a vascular access set. The vascular access set can be, for example, a V-STICK vascular access set for placement of 0.035″ or 0.038″ guidewires into the vascular system using small needle access to reduce puncture site and vessel trauma. The set can include a coaxial dilator, (e.g., a 4 F×10 cm or 5 F×10 cm dilator), a 21 G×7 cm needle (optionally with echogenic tip), and a guidewire.

Trocars

The coating can be applied to trocars, such as, for example, a CVP feeding trocar, or a CVA feeding trocar, such as those available from American Medical Instruments, Inc.

Huber Needles in Cannula Form

The coating can be applied to a Huber needle, e.g., in cannula form, straight or bent (e.g., with a 90° bend) for use in continuous, portal, and intravenous drug therapy. Suitable Huber needles are available from American Medical Instruments, Inc.

Epidural Needles

The coating can be applied to an epidural needle, such as a Tuohy or Hustead epidural needles as well as side port pencil point or standard spinal needles. Suitable epidural needles are available from American Medical Instruments, Inc.

Catheter Fixation Devices

The coating can be applied to a surface of a catheter fixation device, such as SKATER-FIX from InterV/PBN Medical.

Drainage Devices

Drainage devices can include a surface coated with a lubricious coating. Suitable drainage devices include drainage catheters, single step drainage catheters, nephrostomy catheters, balloon catheters, biliary drainage catheters, PTC catheter needles, or PTCD biliary stents.

For example, the drainage catheters can be SKATER® drainage catheters (described above) or a TCD drainage catheter (optionally with a safety string lock system) or a TCD single step drainage catheter (optionally with a safety string lock system). The TCD catheters are available from PBN Medical. The TCD drainage catheter can have dimensions of 4 F×20 cm (which accepts a 0.021″ guidewire), 5.7 F×20 cm, 5.7 F×30 cm, 7 F×20 cm, 7 F×30 cm, (which accept a 0.035″ guidewire) or dimensions of 8.4 F×20 cm, 8.4 F×30 cm, 10 F×20 cm, or 10 F×30 cm (which accept a 0.038″ guidewire). A TCD single step drainage catheter can have dimensions of, for example, 5.7 F×20 cm, 5.7 F×30 cm (which use a 19 G trocar), 7 F×20 cm, 7 F×30 cm, (which use a 18 G trocar) or dimensions of 8.4 F×20 cm, 8.4 F×30 cm, 10 F×20 cm, or 10 F×30 cm (which use a 17 G trocar).

A nephrostomy catheter can be, for example, a SKATER® nephrostomy catheter, optionally with a locking pigtail. The catheter can have dimensions of 6 F, 7 F, 8 F, 10 F, 12 F, or 14 F, with a length of 25 cm to 35 cm. The catheter can accept a 0.035″ guidewire (e.g. for 6 F and 7 F catheter) or a 0.038″ guidewire (e.g., 8 F and higher catheters). The nephrostomy catheter can be a pigtail catheter with a curved portion (the “pigtail”) including sideholes. The nephrostomy catheter can be a silicone balloon catheter for long term nephrostomy. The silicone balloon catheter can have dimensions of 6 F×25 cm (for use with a 0.028″ guidewire and a 12 F split sheath), 8 F×35 cm (for use with a 0.038″ guidewire and a 13 F split sheath), 10 F×35 cm (for use with a 0.038″ guidewire and a 14 F split sheath), or 12 F×35 cm (for use with a 0.038″ guidewire and a 16 F split sheath).

A biliary drainage catheter can be, for example, a SKATER® biliary drainage catheter (e.g., with locking or non-locking pigtail), or a polyethylene biliary drainage catheter. The SKATER® biliary drainage catheter can have dimension of 8 F×40 cm, 10 F×40 cm, or 12 F×40 cm, and use a 0.038″ guidewire and a metal and flexible stiffening cannula. The polyethylene biliary drainage catheter (optionally with pigtail) can have dimensions of 5 F×50 cm, 6.6 F×50 cm, 7 F×50 cm, 8.4×50 cm, or 10 F×60 cm, and accept a 0.035″ guidewire, and include 8 or 10 sideholes. A tapered ring polyethylene biliary drainage catheter can have dimensions of 6.6 F to 10 F, a length of 40 cm to 50 cm, and include 6 to 32 sideholes. Suitable biliary drainage catheters are available from InterV/PBN Medical.

A PTC catheter needle can have dimensions of, for example, 5 F or 5.7 F with a length of 17 cm or 25 cm. The needle can have a slanting point stylet with 19° bevel; a trocar point stylet; or be a single-packed catheter. The catheter can accept a 0.035″ or 0.038″ guidewire.

A PTCD biliary stent can have dimensions of 6 mm to 10 mm in diameter with a length of 40 mm to 100 mm. The stent can be placed with an introducer, which can have dimensions of 8 F×44 cm. A surface of the introducer or the stent can include a coating.

Introducers and Needles

The coating can be applied to a surface of an introducer or a needle, such as, for example, a SKATER® Introducer, a SKATER® centesis catheter needle (described above), an access needle, or a sheathed introducer needle. A SKATER® Introducer is a system for simple, accurate and atraumatic placement of a 0.035″ or 0.038″ guidewire in non-vascular procedures.

Access needles can have, e.g., a puncture needle with stylet; a PTFE coated puncture needle; or a trocar needle. The access needle can have dimensions ranging from 17 G to 21 G with a length ranging from 10 cm to 33 cm.

Sheathed introducer needles can include, for example, sheathed Chiba introducer needles (e.g., for aspiration or as an intermittent link to a larger drainage catheter), a sheathed puncture needle with stylet (e.g., for aspiration of fluid and guidewire introduction), or an introducer sheath/needle with a trocar point (e.g., to provide an access path for a guidewire). Sheathed introducer needles can have dimensions, for example, of 19 G to 22 G and 15 cm to 20 cm.

Non-Vascular Guidewires

The coating can be applied to a non-vascular guidewire. The non-vascular guidewire can be a nitinol guidewire or a stainless steel guidewire, (which can have dimensions of 0.018″×80 cm, optionally with a 6.5 cm radiopaque coil tip), an Amplatz WORKER guidewire (e.g., a stainless steel guidewire with flat wire construction and a PTFE coating, with dimensions of 0.035″ and a length of 80 cm to 180 cm, optionally with a 3.5 cm or 7.5 soft tip, and optionally with a J curve), or a Lunderquist stainless steel guidewire (which can be stiffer than other guidewires, but with a malleable tip, and have dimensions of 0.028″ or 0.035″ and a length of 80 cm to 120 cm, a 7.5 cm flexible coil, and optional J curve).

Dilators

The coating can be applied to a dilator, such as, for example, a screw dilator, a screw dilator with introducer split sheath, or other dilators. A screw dilator can atraumatically separate tissue for dilation up to 14 F with a single dilator. Screw dilators can have dimensions ranging from 7 F to 18 F and a length of 25 cm. A dilator with introducer split sheath allows free passage of a catheter, and the sheath can be easily removed by peeling it apart. Screw dilators with introducer split sheaths can have dimensions ranging from 7 F to 16 F and a length of 25 cm.

Vascular Guidewires

The coating can be applied to a vascular guidewire. The vascular guidewire can be, for example, a CanaliZer® hydrophilic guidewire, a WORKER® guidewire, an Amplatz WORKER® guidewire, a POINTER® nitinol guidewire, a PLACER® guidewire, or a FLEXER® hydrophilic guidewire.

A CanaliZer®G hydrophilic guidewire can have a polyurethane-coated nitinol wire which can be standard or stiff, and can be straight or curved. The guidewire can have a diameter of 0.035″ or 0.038″ and a length ranging from 80 cm to 260 cm.

A WORKER® guidewire can have a straight or J-curved tip, and can have a diameter of 0.025″, 0.035″, or 0.038″, and a length ranging from 150 cm to 400 cm. The guidewire can have a PTFE coating.

An Amplatz WORKER® guidewire can have both an extra stiff shalt and an atraumatic tip, with a flat wire construction. The guidewire can have a diameter of 0.035″ and a length ranging from 90 cm to 180 cm, and can optionally have a 3.5 cm or 7 cm soft tip, and optionally have a J-curved tip.

A POINTER® guidewire can have a nitinol guidewire with a radiopaque coil tip, which can have a hydrophilic coating. The guidewire can have a diameter of 0.018″ or 0.020″, a length ranging from 200 cm to 300 cm, and a coil tip of 4 cm to 6 cm.

A PLACER® guidewire can have a PTFE coated stainless steel core, with a radiopaque coil tip. The guidewire can have a diameter of 0.018″ or 0.020″, a length ranging from 200 cm to 300 cm, and a coil tip of 4 cm to 6 cm.

Repositionable Localization Needles

The coating can be applied to a repositionable localization needle (e.g., for use in conjunction with mammography). The repositionable localization needle can be, e.g., a Hawkins I or Hawkins II needle, or a Horner Mammalok® needle.

A Hawkins I needle can include a retractable side barb to lock the needle in place, or, when retracted, to allow repositioning. The needle can include centimeter markings for depth placement, and a lock-down disk to stabilize the needle. The needle can be a 20 gauge needle with a length ranging from 5 cm to 12.5 cm.

A Hawkins II needle can be a traditional “hardwire” needle, or a “cable,” i.e., a strand of smaller wires, which can provide greater flexibility. The needle can include markings for depth placement, a skin retention clip to stabilize the needle. The needle can be a 20 gauge needle with a length ranging from 5 cm to 12.5 cm.

A Horner Mammalok® needle can have a retractable, repositionable, flexible J wire. The needle can include centimeter markings for depth placement, and a stabilizer to stabilize the needle. The needle can be a 20 gauge needle with a length ranging from 5 cm to 12.5 cm.

Localization Needles

The coating can be applied to a localization needle (e.g., for use in conjunction with mammography). The localization needle can be, for example, a Hawkins III needle, a “D” wire breast localization needle, an ACCURA breast localization needle, or an ACCURA II breast localization needle.

A Hawkins III needle can be a traditional “hardwire” needle, or a “cable,” i.e., a strand of smaller wires, which can provide greater flexibility. The needle can include markings for depth placement, and an end hole for dye injection or fluid aspiration. The needle can be a 20 gauge needle with a length ranging from 3 cm to 12.5 cm.

A “D” wire breast localization needle can have a “D”-shaped cross-section. The needle can include markings for depth placement, a skin retention clip to stabilize the needle, and an end hole for dye injection or fluid aspiration. The needle can be a 20 gauge needle with a length ranging from 3 cm to 15 cm.

An ACCURA breast localization needle can have a springhook hardwire with stiffener design. The needle can include markings for depth placement, a skin retention clip to stabilize the needle, and an end hole for dye injection or fluid aspiration. The needle can be a 20 gauge or 21 gauge needle with a length ranging from 3 cm to 10 cm.

An ACCURA II breast localization needle can have a springhook hardwire with stiffener design, and can be used with a 23 G stiffening cannula. The needle can include markings for depth placement, a skin retention clip to stabilize the needle, and an end hole for dye injection or fluid aspiration. The needle can be a 20 gauge needle with a length ranging from 5 cm to 12.5 cm.

Special Radiology

The coating can be applied to a surface of devices used in special radiology applications. For example, the coating can be applied to devices used in galactography/sialography, to enteroclysis and duodenography catheters, to devices in a TearLeader® stent set, to an HSG catheter, to devices in a fallopian tube set or a cholangiography set, or a Quadra-Fuse multi-pronged injection needle.

Galactography/sialography devices can include a polyethylene catheter or fine blunt cannula, which can be straight, curved, or bent; and a dilator.

Enteroclysis and duodenography catheters can be made of polyvinyl chloride and include an inflatable antireflux balloon. The catheters can have a soft, round tip for atraumatic introduction.

A TearLeader® stent set is used for placing a stent in the nasolacrimal duct. It can have an “S” shape for single step placement. The set can include a 3 F×10 cm dacryocystography catheter with ball tip stylet, a 0.018″ nitinol guidewire, and a 6 F×4.5 cm S-shaped stent with side holes.

A HSG catheter can be used, for example, for radiological hysterosalpingography or hydro hysterosonography. The catheter can include a soft, non-latex balloon which prevents leakage of saline or contrast media. The catheter can have dimensions of, for example, 5 F×40 cm or 7 F×40 cm.

Devices in a fallopian tube set (e.g., for use in salpingography and fallopian tube procedures) can include a 10 F balloon catheter, a 5 F selective salpingography catheter, and/or (for uterine corunal access) a 3 F radiopaque catheter and a nitinol 0.018″ guidewire.

A cholinangiography set can include an 18 G×6.5 cm blunt curved needle with 25 cm connecting tube, and can be used for contrast injection while reducing the risk of puncture of the common bile duct or choledochus.

Vascular Access

The coating can be applied to a device used for vascular access, such as, for example, a guidewire introducer needle, or a vascular dilator. A guidewire introducer needle can be used in an anterior, single-wall arterial/Seldinger percutaneous procedure. The needle can have a non-coring “B” arterial bevel, and an optional winged base plate. The needle can be an 18 G×7 cm needle (for 0.038″ guidewires) or a 19 G×7 cm needle (for 0.035″ guidewires). The needle can be a modified Potts/Cournand needle (e.g., for carotid angiography, direct arterial pressure monitoring, blood sampling, or percutaneous catheterization).

A vascular dilator can be a radiopaque polyethylene dilator, with a smooth rounded tip, and a Luer lock hub for contrast injection and/or guidewire exchange. The dilator can have dimensions of 4 F to 8 F and a length of 20 cm. It can accommodate a 0.035″ or 0.038″ guidewire.

Tissue Access

The coating can be applied to a device used for tissue access, such as, for example, a trocar used to insert a continuous glucose monitor. The trocars can be used with launching devices that are designed to quickly insert various monitoring and other devices by spring-loaded action, thus minimizing pain to the patient. The coating can also be applied to various administration needles, such as, for example, butterfly needles that are typically inserted manually into patients. The solution may be applied as part of the manufacturing process, or just prior to insertion into the patient. A few moments to air dry should be allowed when applied just prior to insertion into the patient.

Angiography Catheters

The coating can be applied to an angiography catheter, such as a GOLDEN-RULE scaling catheter, which can include radiopaque bands at regular intervals for vessel lumen sizing prior to aortic graft placement, angioplasty, and other interventional procedures. The catheter can have dimensions of 5 F×90 cm, include a pigtail and 21 radiopaque markers (e.g., gold rings), 6 side holes, accept a 0.035″ (0.89 mm) guidewire, and accept a maximum injection pressure of 1200 psi; or can have dimensions of 5 F×65 cm with 11 gold rings, 8 side holes, accept a 0.035″ (0.89 mm) guidewire, and maximum pressure 1200 psi.

Urology Devices

The coating can be applied to devices used in urological procedures, such as, for example, a ureteral pigtail stent set, catheters, guidewires, or a TRU-CORE I Uro biopsy needle/instrument.

A ureteral pigtail stent set can include an introducer (with a transparent inner catheter and a positioning catheter), a torque handle for manipulating a guidewire, a guidewire (such as a PTFE coated WORKER® guidewire), and the ureteral stent. The stent can be a soft polyurethane with a size of 6 F to 8 F and a length of 24 cm to 28 cm.

Urological accessories, such as catheters and guidewires, can be coated. The catheter can be a pyelography catheter (e.g., with end hole design and centimeter markings); the guidewire can be, for example, a WORKER® or a SURFER® guidewire.

A TRU-CORE I Uro biopsy needle can be an 18 G needle with a 19 mm sample notch, with centimeter markings and an echogenic tip.

Oncology Devices

The coating can be applied to devices used in oncology procedures, such as, for example, a bone marrow biopsy needle, a bone marrow aspiration needle, a bone morrow access needle, a PSS prostate seeding set, or a prostate stabilization set. The bone marrow biopsy needle can be, e.g., a SNARE-LOK needle, or a T-LOK needle.

A PSS prostate seeding needle can be a disposable needle used to facilitate transperineal, radioactive seed implant procedures. The needle can have an echogenic tip for visualization under ultrasound guidance. The needle can have centimeter and half-centimeter markings. A prostate stabilization set can include a needle with a side barb to immobilize the prostate gland during transperineal seeding procedures. The needle can have an echogenic tip.

Stenting

The coating can be applied to a stent or devices used in stenting, such as, for example, a pigtail biliary stent, a guidewire and guiding/pusher catheters for biliary stenting, or a stent introducer system and sizeguide catheter.

A pigtail biliary stent can have a pigtail at each end, each having five sideholes, and can be made of radiopaque polyethylene. It can have dimensions of 7 F to 10 F, with a total length ranging from 6.5 cm to 17.5 cm. The stent can be placed using a guidewire (e.g., a WORKER® guidewire or a hydrophilic coated stainless steel guidewire) and an introducer system (e.g., a 7 F or 10 F biliary stent introducer system).

A guiding/pusher catheter can be, e.g., a 5 F or 6 F PTFE catheter, optionally with a radiopaque band (e.g., of gold); or can be a radiopaque FEP catheter, with a size ranging from 7 F to 11.4 F.

A stent introducer system includes an inner and outer catheter. The inner catheter is made of radiopaque PTFE and the outer catheter is made of radiopaque FEP. The inner catheter has three markers for enhanced visualisation. The inner catheter can have a size of 5 F or 6 F, and can be paired with an outer catheter of size 8.5 F or 10 F, respectively.

A sizeguide catheter can be made of radiopaque PTFE and have 3 distance markers, which are used as a reference point for the radiographic magnification, thus facilitating measurement of the stricture to select the proper stent size. The catheter can have a removable hub for contrast injection, and can also be used as a guiding catheter.

Stone Removal

The coating can be applied to a device for stone removal, e.g., removal of stones from the common bile duct, such as a catheter. The stone removal catheter can be tapered, e.g., having a diameter of 5 F at the distal end and 7 F at the proximal end. The distal end can include an inflatable balloon, with a radiopaque band under the balloon for visualization. The catheter can have dimensions of 7 F (tapered to 5 F at the distal end)×200 cm, and accept a 0.035″ guidewire. An exemplary stone removal catheter is the EXPEL catheter from PBN Medicals.

Nasal Duct

The coating can be applied to a nasal bile catheter, e.g., for drainage or infusion of the nasal bile duct. The nasal bile catheter can be curved at the distal end to prevent catheter slip out. The catheter can be a 7 F catheter having 10 side holes and for use with a guidewire (e.g., a 0.035″ coated guidewire). The coating can also be applied to a nasal tube (e.g., of soft polyvinylchloride) for guiding the nasal catheter from the mouth out through the nose.

Colon Decompression

The coating can be applied to a colon decompression catheter, e.g., for colonscopic decompression in toxic megacolon, pseudo obstruction and decompression of the colon proximal to a stricture. The catheter can be made of radiopaque polyvinylchloride, have dimensions of 16 F or 18 F×175 cm, have side holes (e.g., six side holes). The catheter can be used with a guidewire, such as a 0.035″ PTFE coated guidewire.

The foregoing medical equipment can be formed of various materials, including organic and inorganic polymers as well as metal, ceramic, or glass. Organic polymers include polymers or copolymers of, for example, polyurethanes, silicones, polyvinylchloride, polyolefins (including high density and low density polyethylene, and polypropylene) polyamides and latex and metals including steel; as well inorganic polymers. The coatings can be applied to the various materials, as required by the construction of the device being coated.

### EXAMPLES

The examples of coating solutions listed below are illustrative and are not intended to be limiting. These compositions are adapted to be used as coatings for mesh, wiry, flat and/or sharp metal surfaces as one or more layers.

| | |
|---|---|
| | Amount |
| Component | (grams) |

Stock Solutions
Stock Solution A

| | |
|---|---|
| Cymel 248-8 | 24.00 |
| Paraloid AT-746 | 76.00 |

Stock Solution B

| | |
|---|---|
| Nitrocellulose, ¼″ RS | 9.00 |
| 4-Butyrolactone | 91.00 |

Stock Solution C

| | |
|---|---|
| Tetrahydrofuran | 95.00 |
| Polyethylene-co-acrylic acid (20% acrylic acid) | 5.00 |

Stock Solution D

| | |
|---|---|
| Epoxy resin (EPOTUF 38-505) | 50.00 |
| Tetrahydrofuran | 50.00 |

Stock Solution E

| | |
|---|---|
| TECOFLEX SG-93A | 10.00 |
| Tetrahydrofuran | 90.00 |

Stock Solution F

| | |
|---|---|
| BUTVAR B98 | 6.25 |
| Isopropanol | 93.75 |

Stock Solution G

| | |
|---|---|
| Stock Solution B | 5.00 |
| Cyclohexanone | 95.00 |

Stock Solution H

| | |
|---|---|
| Nitrocellulose ¼″ RS | 25.20 |
| Toluene | 11.30 |
| n-Butyl acetate | 17.00 |
| Ethyl acetate | 34.80 |
| Dibutylphthtalate | 6.60 |
| Camphor | 4.80 |
| UVINUL M40 | 0.30 |

Stock Solution I

| | |
|---|---|
| Stock Solution B | 1.00 |
| Cyclohexanone | 99.00 |

Stock Solution J

| | |
|---|---|
| Stock Solution C | 73.50 |
| Cyclohexanone | 19.60 |
| Stock Solution A | 2.90 |
| Tetrahydrofuran | 3.00 |
| Trichloroacetic acid | 1.00 |

Stock Solution K

| | |
|---|---|
| Diisocyanate (Tycel 7351) | 78.00 |
| Tetrahydrofuran | 22.00 |

Coating Solutions
Coating Solution A

| | |
|---|---|
| n-Butyl acetate | 42.26 |
| Toluene | 25.36 |
| Nitrocellulose ¼″ RS | 3.84 |
| Dibutylphthalate | 15.48 |
| Camphor | 2.85 |
| Polyisocyanate (TYCEL 7000) | 8.01 |
| Stock Solution A | 1.98 |
| 2-hydroxy-4-methoxy benzophenone | 0.22 |

Coating Solution B

| | |
|---|---|
| Ethanol | 44.05 |
| Isopropanol | 19.93 |
| 4-Butyrolactone | 20.01 |
| MEK | 2.77 |
| Polyvinylpyrrolidone (PVP K90) | 5.21 |
| Acetic acid | 6.21 |
| Polyethylene glycol (MW 400) | 1.63 |
| Stock Solution B | 0.19 |

Coating Solution C

| | |
|---|---|
| Stock Solution C | 78.21 |
| Stock Solution D | 2.31 |
| Cyclohexanone | 15.68 |
| Aromatic polycarbonate-based polyurethane solution (22-25% by | 3.80 |
| weight in DMAC) | |

Coating Solution D

| | |
|---|---|
| Aromatic polycarbonate-based polyurethane solution (22-25% by | 31.50 |
| weight in DMAC) | |
| Cyclohexanone | 7.00 |
| Benzyl alcohol | 3.60 |
| Tetrahydrofuran | 17.40 |
| Stock Solution H | 38.00 |
| Iron Blue Dilution | 1.00 |
| TiO₂ Dilution | 1.50 |

Coating Solution E

| | |
|---|---|
| Ethanol | 37.50 |
| Benzyl alcohol | 34.80 |
| Isopropanol | 17.40 |
| Cyclohexanone | 2.70 |
| Polyvinylpyrrolidone (PVP K90) | 5.80 |
| Stock Solution B | 0.01 |
| Polyethylene glycol (MW400) | 1.80 |

Coating Solution G

| | |
|---|---|
| Aromatic polycarbonate-based polyurethane solution (22-25% by | 32.31 |
| weight in DMAC) | |
| Cyclohexanone | 7.18 |
| Benzyl alcohol | 3.69 |
| THF | 17.85 |
| Stock Solution H | 38.97 |

Coating Solution H

| | |
|---|---|
| Toluene | 6.80 |
| MEK | 25.70 |
| Dibutylphthlate | 2.70 |
| Stock Solution A | 3.20 |
| ¼″ RS Nitrocellulose | 6.20 |
| Stock Solution E | 44.00 |
| Trichloroacetic acid | 0.04 |
| THF | 11.40 |

Coating Solution I

| | |
|---|---|
| Ethanol | 10.10 |
| Benzyl alcohol | 18.10 |
| Cyclohexanone | 47.10 |
| Tetrahydrofuran | 22.40 |
| Stock Solution B | 0.20 |
| PVP K90 | 2.10 |

Coating Solution J

| | |
|---|---|
| Toluene | 7.50 |
| Benzyl Alcohol | 7.70 |
| Tetrahydrofuran | 12.60 |
| Cyclohexanone | 10.00 |
| Dibutylphthalate | 3.00 |
| Stock Solution A | 3.50 |
| Nitrocellulose, ¼″ RS | 6.90 |
| Stock Solution E | 48.70 |
| Trichloroacetic acid | 0.04 |

Coating Solution K

| | |
|---|---|
| Stock Solution H | 38.90 |
| Cyclohexanone | 20.10 |
| Benzyl alcohol | 11.00 |
| Stock Solution E | 19.60 |
| Stock Solution A | 10.30 |
| Trichloroacetic acid | 0.10 |

Coating Solution L

| | |
|---|---|
| Ethanol | 72.00 |
| 4-Butyrolactone | 16.80 |
| PVP K90 | 6.60 |
| Stock Solution I | 4.60 |

Coating Solution M

| | |
|---|---|
| Ethanol | 36.60 |
| Benzyl alcohol | 32.50 |
| Isopropanol | 16.20 |
| PVP K90 | 4.10 |
| Acetic acid | 8.10 |
| Stock Solution G | 2.50 |

Coating Solution O

| | |
|---|---|
| Stock Solution J | 98.50 |
| Stock Solution K | 1.50 |
| Coating Solution Q | |
| THIXON 422 | 50.00 |
| THIXON 917 | 50.00 |

Coating Solution R

| | |
|---|---|
| Toluene | 7.5 |
| Benzyl alcohol | 7.7 |
| THF | 12.6 |
| Cyclohexanone | 10.0 |
| Dibutylphthlate | 3.0 |
| Stock Solution A | 3.5 |
| Nitrocellulose ¼″ RS | 6.9 |
| Stock Solution E | 48.8 |

Coating Solution S

| | |
|---|---|
| Stock Solution C | 81.30 |
| Stock Solution D | 2.40 |
| Cyclohexanone | 16.30 |
| Coating Solution T | |
| Toluene | 7.51 |
| Butyl acetate | 7.71 |
| Tetrahydrofuran | 12.61 |
| Cyclohexanone | 10.01 |
| Dibutyl phthalate | 3.00 |
| Stock Solution A | 3.50 |
| Nitrocellulose, ¼″ RS | 6.91 |
| Stock Solution E | 48.75 |

Coating Solution U

| | |
|---|---|
| Ethanol | 75.4 |
| 4-Butyrolactone | 17.6 |
| Polyvinylpyrrolidone (PLASDONE K-90) | 5.0 |
| Stock Solution B | 0.02 |
| Cyclohexanone | 1.98 |

Coating Solution V

| | |
|---|---|
| Butyl acetate | 55.37 |
| Dibutyl phthalate | 2.64 |
| Stock Solution A | 3.19 |
| Nitrocellulose, ¼″ RS | 6.01 |
| Stock Solution E | 21.52 |
| Pigment paste | 11.27 |

Coating Solution W

| | |
|---|---|
| Ethanol | 35.31 |
| Benzyl alcohol | 31.32 |
| Isopropanol | 15.66 |
| Cyclohexanone | 2.29 |
| Glacial acetic acid | 7.88 |
| Polyvinylpyrrolidone (PLASDONE K-90) | 5.49 |
| Stock Solution B | 0.20 |
| Polyethylene glycol (MW 400) | 1.70 |
| Stock Solution F | 0.15 |

Coating Solution X

| | |
|---|---|
| Polyvinylpyrrolidone (360K) | 1.25 |
| Denatured ethanol | 6.75 |
| Benzyl alcohol | 1.20 |
| Cyclohexanone | 2.765 |
| Tetrahydrofuran | 31.50 |
| RS nitrocellulose, ¼ second | 0.003 |
| 4-Butyrolactone | 0.032 |

Coating Solution Y

| | |
|---|---|
| Water | 11.03 |
| Denatured ethanol | 5.02 |
| Polyethylene glycol (MW 400) | 0.16 |
| Polyethylene glycol (MW 8000) | 1.49 |

Coating Solution Z

| | |
|---|---|
| Polyvinylpyrrolidone (360K) | 0.35 |
| Denatured ethanol | 1.88 |
| Benzyl alcohol | 3.36 |
| Cyclohexanone | 6.42 |
| Hydroxyl function acrylic polymer (PARALOID AT 63 from | 0.10 |
| Rohm & Haas) | |
| Xylene | 0.10 |

Coating Solution AA

| | |
|---|---|
| Polyvinylpyrrolidone (360K) | 0.49 |
| Denatured ethanol | 1.88 |
| Benzyl alcohol | 3.36 |
| Cyclohexanone | 6.41 |
| Hydroxyl function acrylic polymer (PARALOID AT 63 from | 0.30 |
| Rohm & Haas) | |

Coating Solution AB

| | |
|---|---|
| Polyvinylpyrrolidone (360K) | 0.49 |
| Denatured ethanol | 1.88 |
| Benzyl alcohol | 3.36 |
| Cyclohexanone | 6.41 |
| Hydroxyl function acrylic polymer (PARALOID AT 63 from | 0.20 |
| Rohm & Haas) | |

Coating Solution AC

| | |
|---|---|
| 10% (w/w) PVP (360K) in denatured ethanol | 4.00 |
| Denatured ethanol | 7.19 |
| 4-Butyrolactone | 2.43 |
| PVP K90 | 0.54 |
| Benzyl alcohol | 2.90 |
| THF | 7.30 |
| Stock Solution B | 0.87 |

Coating Solution AD

| | |
|---|---|
| Polyamide resin | 0.18 |
| Epoxy resin | 0.10 |
| Tetrahydrofuran | 7.02 |
| Dimethylacetamide | 1.00 |
| PVP (360K) | 0.49 |
| Denatured ethanol | 4.41 |

Coating Solution AE

| | |
|---|---|
| Dibutyl phthalate | 0.73 |
| Denatured alcohol | 4.27 |
| Ethyl acetate | 0.97 |
| Toluene | 1.65 |
| Nitrocellulose, ¼ sec. RS | 1.62 |
| Penn Blue | 0.28 |
| Penn White | 0.51 |
| Penn Brown | 0.17 |
| Benzyl alcohol | 1.80 |
| PARALOID AT 51 acrylate resin | 0.88 |

Coating Solution AF

| | |
|---|---|
| Polyvinylpyrrolidone (360K) | 0.70 |
| Denatured ethanol | 4.50 |
| Glacial acetic acid | 1.00 |
| Benzyl alcohol | 4.00 |
| Isopropyl alcohol | 2.00 |
| Stock Solution G | 0.31 |

Coating Solution AG

| | |
|---|---|
| Polyethylene based copolymer | 1.68 |
| THF | 15.54 |
| DMAC | 19.87 |
| Anisole | 21.27 |
| Xylenes | 41.34 |
| Epoxy polymer | 0.33 |

Coating Solution AH

| | |
|---|---|
| Aromatic polycarbonate-based polyurethane solution (22-25% by | 11.03 |
| weight in DMAC) | |
| Anisole | 20.22 |
| MIBK | 68.48 |
| DMAC | 0.27 |

Coating Solution AI

| | |
|---|---|
| Aromatic polycarbonate-based polyurethane solution (22-25% by | 9.16 |
| weight in DMAC) | |
| Nitrocellulose, H-15 Grade | 1.38 |
| Anisole | 27.65 |
| MEK | 30.00 |
| DMAC | 11.81 |
| n-Butanol | 20.00 |

Coating Solution AJ

| | |
|---|---|
| Ethanol | 39.90 |
| Benzyl alcohol | 35.40 |
| Isopropanol | 17.60 |
| PVP K90 | 4.40 |
| Stock Solution G | 2.70 |

Coating Solution AL

| | |
|---|---|
| Ethanol | 34.10 |
| Benzyl Alcohol | 30.20 |
| Isopropanol | 15.10 |
| Stock Solution G | 9.30 |
| Acetic Acid | 7.50 |
| PVP K90 | 3.80 |

Coating Solution AM

| | |
|---|---|
| Stock Solution C | 81.30 |
| Stock Solution D | 2.40 |
| Cyclohexanone | 16.30 |

Coating Solution AN

| | |
|---|---|
| Denatured anhydrous ethanol | 31.66 |
| Benzyl alcohol | 22.75 |
| Cyclohexanone | 42.35 |
| Stock Solution B | 0.07 |
| Polyvinylpyrrolidone K-90 (PVP K-90) | 3.17 |

### Example 1

A stainless steel blade (SHARPOINT Ophthalmic Slit Blade from Surgical Specialties Corporation, Reading, Pa.) was dip coated with the following primer solution and dried in an oven for 15 minutes at 120° C.

Coating Solution 1

| Component | Amount (grams) |
|---|---|
| Ethylene acrylic acid copolymer | 1.68 |
| Epoxy resin | 0.44 |
| Tetrahydrofuran (THF) | 15.54 |
| Dimethyl acetamide (DMAC) | 19.86 |
| Anisole | 21.27 |
| Xylenes | 41.21 |

The sample was then dip coated with the following solution and dried in an oven for 15 minutes at 120° C.

Coating Solution 2

| Component | Amount (grams) |
|---|---|
| Aromatic polycarbonate-based polyurethane | 9.16 |
| solution (23% in DMAC) | |
| Nitrocellulose | 1.38 |
| Dimethyl acetamide (DMAC) | 11.81 |
| Anisole | 27.65 |
| Methyl ethyl ketone (MEK) | 30.00 |
| n-Butanol | 20.00 |

The sample was then dip coated with the one of the following hydrophilic polymer solutions.

| | |
|---|---|
| Component | Amount (grams) |

Coating Solution 3

| | |
|---|---|
| Polyvinylpyrrolidone (PVP) | 4.01 |
| Nitrocellulose | 0.0054 |
| 4-Butyrolactone | 0.06 |
| Ethanol | 10.31 |
| Benzyl alcohol | 18.43 |
| Cyclohexanone | 44.56 |
| Isopropanol | 22.63 |

Coating Solution 4

| | |
|---|---|
| Polyvinylpyrrolidone (PVP) | 3.99 |
| Nitrocellulose | 0.03 |
| 4-Butyrolactone | 0.33 |
| Ethanol | 10.28 |
| Benzyl alcohol | 18.37 |
| Cyclohexanone | 44.43 |
| Isopropanol | 22.56 |

Coating Solution 5

| | |
|---|---|
| Polyvinylpyrrolidone (PVP) | 3.03 |
| Nitrocellulose | 0.009 |
| 4-Butyrolactone | 0.091 |
| Ethanol | 10.41 |
| Benzyl alcohol | 18.60 |
| Cyclohexanone | 45.01 |
| Isopropanol | 22.85 |

The coatings were insoluble in water and were lubricious when wet. SEM photographs of the blade tips revealed that there was no build up of coating material at the knife blade edge (data not shown).

### Example 2

### Dye Uniformity Test

The thickness and uniformity of the coating can be measured using the following procedure. Dip the blade into Gentian Violet dye solution. Rinse excess dye off the substrate by holding it under running cold water. The rinsing can be stopped when the water running off is clear and no more dye appears to be washing off the coated article. Visually inspect the dyed section. The intensity of the color of the dye is a function of the thickness of the coating (i.e., the thinner the coating, the lighter the dye intensity). The sample should look uniform without voids or extra dark or light regions in the coated area. The entire coated surface should be uniformly covered with dye for it to pass the dye uniformity test.

Five blade samples were coated with Coatings 1, 2, and 3 using the procedure described in Example 1 and tested in the dye uniformity test. All five blades tested passed. Two additional blades coated with Coatings 1, 2, and 4 or 5 also passed the dye uniformity test.

### Example 3

### Wet Abrasion Test

Fold a piece of brown paper towel into fourths and completely saturate it with water until water is dripping from the paper towel. Hold the piece of water soaked brown paper towel in one hand, lightly rub the dyed substrate (dyed with Gentian Violet as described in Example 2) between the index finger and thumb using almost no pressure, for 50 cycles (one cycle=a stroke up and down the substrate). If the dye does not fade or fades only slightly, the sample passes the wet abrasion test. In some cases, the dye may fade but there is no loss of lubricity. In such cases, if the dye completely fades from the substrate, the sample may be re-dyed and rinsed, and then re-checked for lubricity as described above. If the topcoat layer accepts the dye, this confirms that the top coating layer is still present. If the sample does not accept the dye, the coating layer may have come off of the sample.

Five blade samples were coated with Coatings 1, 2, and 3 using the procedure described in Example 1 and tested in the wet abrasion test. All five blades tested passed. Two additional blades coated with Coatings 1, 2, and 4 or 5 also passed the wet abrasion test.

### Example 4

### Dry Adhesion Test

Score the device with a razor blade by scraping a 1-2 mm wide section of the coating down to the bare metal. Using Type 810 SCOTCH brand tape (3M Company, St. Paul, Minn.), cover the substrate making sure the tape covers the scored section. Firmly press the tape over the substrate with pad of fingertip (approximately 5-6 presses). Briskly pull the tape off the substrate at a 180-degree angle. Examine the tape and substrate for evidence of peeled or removed coating. If the tape is clean of coating and the substrate coating remains smooth and intact the coating passed dry adhesion test. If coating is present on the tape or the coating has peeled or blistered on the substrate, the substrate fails the dry adhesion test.

Five blade samples were coated with Coating Solutions 1, 2, and 3 using the procedure described in Example 1 and tested in the dry adhesion test. All five blades tested passed. Two additional blades coated with Coating Solutions 1, 2, and 4 or 5 also passed the dry adhesion test.

### Example 5

### Coating Thickness Measurements of Knives Coated with Hydrophilic Polymer Solutions of Varying Viscosities

A total of 13 blades were coated with Coating Solutions 1 and 2 using the procedure of Example 1. Two 200 g aliquots of Coating Solution 3 were further diluted with a solvent mixture having the same solvent ratios as in Coating Solution 3 to provide three different coating solutions of varying viscosity: 54 cps (Coating Solution 3); 45 cps (Coating Solution 4); and 32 cps (Coating Solution 5). Each of the samples was coated with one of Solution 3, 4, or 5. The final coating thickness for each of the 13 knives was measured using a TENCOR profilometer.

TABLE 1
Coating Thickness Measurements

| Number of Samples | | |
|---|---|---|
| Tested | Hydrophilic Coating | Coating Thickness (μm) |
| 6 | 3 (52 cps) | 5.3-10.3 |
| 4 | 4 (45 cps) | 4.8-6.5 |
| 3 | 5 (32 cps) | 5.1-6.8 |

### Example 6

Stainless steel taper point and cut point suture needles (0.026×4.5 cm straight needles) from Surgical Specialties Corporation, Reading, Pa.) were coated using Coating Solutions A, B, and C, as described in Example 1. The coatings were insoluble in water and were lubricious when wet.

Other types of needles made entirely or partially from stainless steel, including biopsy needles, breast localization needles, injection needles, bone marrow aspiration, access, and harvest needles, biopsy introducer needles, and epidural needles may be coated with these formulations, as well.

### Example 7

A polyurethane clad guidewire from PBN Medicals/InterV (Denmark) was dip coated with a primer solution (Coating Solution A) and dried in an oven for 30 minutes at 85° C. The sample then was coated with a hydrophilic polymer solution (Coating Solution B).

The coatings were insoluble in water and were lubricious when wet. Coating components were identified to minimize the presence of pigments which are frequently found in many commercial polymeric resins. The solvent systems also were optimized to minimize the use of unnecessary or hazardous solvents, to enhance compatibility of the coating solution with the primer layer and/or substrate, and to improve overall biocompatibility. The solvent systems used in the primer and top coat layer formulations were found to be compatible with the polyurethane cladding material and did not damage or degrade the cladding material.

### Example 8

Coating solutions are described that may be used to coat medical devices made entirely or partially from stainless steel, including guidewires (e.g., guidewires having a coiled portion), stents (vascular and non-vascular), scalpels, blades, suture needles, biopsy needles, and introducer needles.

A stainless steel guidewire having a coiled portion was dip coated with a primer solution (Coating Solution C) and dried in an oven for 30 minutes at 120° C. The sample then was coated with Coating Solution D and dried at 30 minutes at 120° C. to form a tie layer. The sample was then dip coated with a hydrophilic polymer solution (Coating Solution E) and dried at 30 minutes at 120° C.

The coatings were insoluble in water and very lubricious when wet. The coating components and solution viscosity were adjusted to produce a relatively thick coating (about 25-30 microns) that bridged that coils of the guidewire. For devices that may not use a bridging coating (e.g., scalpels, blades, straight or tapered wire portion of guidewires), similar formulations of lower viscosity may be used. The coating adhered well to both straight and coiled portions of the guidewire and did not crack when the coil was flexed. The enhanced flexibility of the coating may be attributed to the presence of polyurethane in the primer and tie layers of the coating. The addition of a PEG component in the hydrophilic top coat formulation improved hydration times and aided in plasticizing the coating and improving its flexibility. The coating was found to be particularly robust and remained free of defects (e.g., pinholes and cracks) even under harsh sterilization conditions (e.g., EtO sterilization in high humidity).

### Example 9

Coating solutions are described that may be used to coat medical devices made entirely or partially from alloys of nickel and titanium (e.g., nitinol) and may be particularly well suited for use with devices that have flexible or mobile components, such as guidewires, guidewires with a coiled portion, and stents (vascular and non-vascular).

A nitinol guidewire was dip coated with a primer solution (Coating Solution C) and dried in an oven for 30 minutes at 85° C. The sample was then dip coated with Coating Solution G and dried in an oven for 30 minutes at 120° C. to form an intermediate tie layer. The sample was then coated with a hydrophilic polymer solution (Coating Solution E) and dried in an oven for 60 minutes at 120° C.

Smooth, uniform coatings were prepared which were insoluble in water and lubricious when wet. Coating components were identified to form a flexible coating which would adhere well to both straight and coiled portions of the guidewire and did not crack when the guidewire was flexed. The addition of a PEG component in the hydrophilic top coat formulation improved hydration times, aided in plasticizing the coating, and improved its flexibility.

### Example 10

Coating solutions are described that may be used to coat devices made entirely or partially from polyester (e.g., PET) threads or filaments (e.g., DACRON), including sutures and fabrics (e.g., surgical meshes).

A polyester fabric was dip coated with Coating Solution H and dried in an oven for 60 minutes at 80° C. The fabric then was dip coated with hydrophilic polymer Coating Solution I and dried for 4 hours at 80° C.

The coating adhered well to the threads, was insoluble in water, and was lubricious when wet. Depending on the type of fabric, a primer layer (e.g., Coating Solution C) may be added to further improve adhesion of the lubricious coating to the threads.

### Example 11

Coating solutions are described that may be used to coat devices made entirely or partially from a fluoropolymer, such as polytetrafluoroethylene (PTFE), fluorinated ethylene propylene (FEP), or poly(vinylidene fluoride) (PVDF). The fluoropolymer may be in the form of a sheet, rod tube, or other shape and may be in a sintered and/or expanded form (e.g., e-PTFE). Representative examples of devices that are made from fluoropolymers include catheters (e.g., delivery catheters for retrieval snares) and introducers.

An expanded PTFE (ePTFE) material was dip coated with Coating Solution H and dried in an oven for 30 minutes at 90° C. The material then was coated with Coating Solution J and dried for 30 minutes at 90° C. The material then was coated with a hydrophilic polymer solution (Coating Solution E) and dried for 2 hours and 90° C.

The coating adhered well to the ePTFE material under wet and dry conditions, was insoluble in water, and was lubricious when wet.

### Example 12

Chemical etching may be utilized to improve adhesion of a lubricious coating to certain types of substrates (e.g., materials with low surface energy such as PTFE, silicone, and other high durometer polymers such as hard nylon and the like.

PTFE tubing (⅛″ thick sidewall) was chemically etched under acidic conditions by dipping in a sodium based etching solution such as FLUOROETCH. The tubing then was dip coated with Coating Solution H and dried for 30 minutes at 85° C. The tubing then was coated with hydrophilic Coating Solution E and dried for 60 minutes at 85° C.

The coating had excellent adhesion to the PTFE tubing, was insoluble in water, and was lubricious when wet.

### Example 13

Coating solutions are described that may be used to coat medical devices made entirely or partially from polymers (e.g., thermoplastic), such as polyimides, polyetheretherketone (PEEK), polytetrafluoroethylene, polyamides, polyetherimides, polycarbonates, polyester and polyether block amides (PBA), polyesters, and styrene-isoprene-styrene (SIS) and styrene-butadiene-styrene (SBS) copolymers. Representative examples of medical devices formed from polymers that may benefit from a lubricious coating include drainage catheters, catheters for delivering retrieval snares, trocars, stents, and cannulae.

Sheets made from polyimide, PEEK, polyether amide, and polyester were cut into strips and coated with a lubricious coating. The polymeric components and solvents in the coating solutions were optimized for the particular polymeric substrate to enhance adhesion of the hydrophilic coating to the substrate. Further, solvent systems were identified which were compatible with the polymeric materials and did not damage or degrade the substrates.

Polyimide

Polyimide tubes (0012″ in diameter×26 cm in length) were dip coated with Coating Solution S and dried for 30 minutes at 85° C. The material then was coated with Coating Solution K and dried for 30 minutes at 85° C. to form a tie layer. The material then was coated with a hydrophilic polymer solution (Coating Solution L) and dried for 60 minutes at 85° C.

Polyetheretherketones (PEEK)

A sample of PEEK sheet cut into strips was dip coated with Coating Solution S and dried for 30 minutes at 85° C. The material then was coated with Coating Solution J and dried for 30 minutes at 85° C. to form a tie layer. The material then was coated with a hydrophilic polymer solution (Coating Solution M) and dried for 60 minutes at 85° C.

Polyether Imide

A sample of ULTEM polyetherimide sheet (General Electric) was cut into strips and dip coated with Coating Solution H and dried for 30 minutes at 85° C. The material then was coated with a hydrophilic polymer solution (Coating Solution U) and dried for 60 minutes at 85° C.

### Example 14

Coating solutions are described that may be used to coat medical devices made entirely or partially from polymers containing amide groups, such as polyamides (e.g., nylon), polyamide copolymers, or polyether block amide polymers (e.g., PEBAX from Arkema, Inc., Philadelphia, Pa.). Representative examples of medical devices formed from polyamide and polyether amide polymers that may benefit from a lubricious coating include drainage and nephrostomy catheters, segmented catheters, catheters for delivering retrieval snares, trocars, and cannulae.

Nylon substrates were coated with a lubricious coating. Depending on the material, adhesion of the coating and/or wet abrasion resistance may be enhanced by treatment with plasma (e.g., oxygen) prior to application of the coating solutions. In particular, coating adhesion to harder materials (i.e., materials having a higher durometer) may benefit from the addition of a plasma treatment.

Samples made from two types of nylon (nylon 11 and nylon 12) were treated with oxygen plasma, coated with a base coat coating solution to form a tie layer, and dried for 30 minutes at 75-85° C. Certain types of nylon substrates may further benefit from the addition of a primer coating after plasma treatment. The nylon samples then were coated with a hydrophilic polymer solution and dried for 60 minutes at 75-85° C.

Nylon materials with hydrophilic coatings

| | | Primer | Tie Layer | |
|---|---|---|---|---|
| | | Coating | Coating | Hydrophilic |
| Substrate | Plasma Treatment | Solution | Solution | Coating Solution |
| Nylon 11 | No | S | J | M |
| Nylon 11 | yes | None | H | U |
| Nylon 12 | yes | None | H | U |
| Nylon 12 | yes | O | J | M |

The coatings adhered well to the nylon substrates, were insoluble in water, and lubricious when wet.

### Example 15

Medical grade polyether block amide polymer samples (e.g., 33 series PEBAX materials from Arkema, Inc., Philadelphia, Pa.) ranging in hardness from 25-72 D were coated with lubricious coatings.

Samples were treated with oxygen plasma, coated with a base coating solution to form a tie layer, and dried for 30 minutes at 75° C. Samples coated with a primer layer after oxygen plasma treatment were dried for 30 minutes at 75° C. Samples then were coated with a hydrophilic polymer solution and dried for 60 minutes at 75° C. The viscosity of the hydrophilic polymer solution ranged from about 30 to 170 cps depending on the desired coating thickness and lubricity.

Polyether block amide polymer materials with hydrophilic coatings

| | | Primer | Tie Layer | |
|---|---|---|---|---|
| | | Coating | Coating | Hydrophilic |
| Hardness (D) | Plasma Treatment | Solution | Solution | Coating Solution |
| 72 | No | Q | R | AJ |
| 72 | Yes | none | H | E |
| 69 | Yes | none | H | E |
| 63 | No | none | H | U |
| 55 | No | none | H | E |
| 40 | No | none | H | E |
| 40 | No | none | H | U |
| 35 | No | none | H | E |
| 25 | No | none | H | E |

Smooth, uniform coatings were prepared (approximately 15 microns) that were water insoluble and lubricious when wet. Adhesion of the lubricious coating to materials was enhanced by use of an oxygen plasma treatment prior to application of the base and top coating solutions. For materials of lower durometer (less than 69 D), adhesion of the hydrophilic coating could be achieved without the use of plasma treatment or a primer coating.

### Example 16

A catheter formed of segments of polyether block amide polymer with hardness values of 35-70 D and nylon 12 was coated with a lubricious coating.

The catheter was treated with oxygen plasma, coated with a base coat Coating Solution J to form a tie layer, and dried for 30 minutes at 75° C. The catheter then was coated with hydrophilic polymer solution (Coating Solution AL) and dried for 60 minutes at 75° C.

Smooth, uniform coatings were prepared (approximately 15 microns) that were water insoluble and lubricious when wet.

### Example 17

A percutaneous transluminal coronary angioplasty (PTCA) balloon made from a polyether-polyamide copolymer was inflated and dip coated with a primer solution (Coating Solution AM) and dried in an oven for 15 minutes at 55° C. The balloon then was coated Coating Solution T and dried in an oven for 30 minutes at 55° C. to form a tie layer. The balloon then was coated with a hydrophilic polymer solution (Coating Solution U) and dried in an oven for 60 minutes at 55° C.

Thin, uniform coatings (approximately 5 microns) were prepared that were water insoluble and lubricious when wet. Coating components were identified that formed a coating which would adhere well to the balloon and conform to the surface such that the surface of the balloon remained free of wrinkles and cracks in both the expanded and unexpanded states. In particular, the use of high boiling solvent (e.g., cyclohexanone) minimized the formation of fine cracks in the coating. The coating did not cause the balloon to contract in the linear direction or reduce balloon length or result in a thickened wall which could adversely reduce the burst pressure of the balloon. In addition, cracks did not form in the coating upon folding of the balloon for insertion into a delivery catheter.

### Example 18

A 5-6 foot length of nitinol guidewire having an aromatic polyurethane cladding was draw coated with a primer solution (Coating Solution V) and dried in an oven for 15 minutes at 85° C. The guidewire then was coated with a hydrophilic polymer solution (Coating Solution W) and dried in an oven for 60 minutes at 85° C.

Water insoluble coatings having a uniform thickness between about 15-25 microns were formed, which were water insoluble and lubricious when wet. The solvent system and coating method were optimized to minimize the amount and use of unnecessary or hazardous solvents, to enhance compatibility of the coating solution with the substrate, and to improve overall biocompatibility. The solvent systems used were found to be compatible with the polyurethane cladding material, such that they did not damage or degrade the cladding material or alter the color of the coating.

### Example 19

A SKATER polyurethane drainage catheter from PBN Medicals/InterV (Denmark) was dip coated with Coating Solution AN and dried in an oven for 60 minutes at 75-80° C.

Water insoluble coatings having a uniform thickness between about 15 and 25 microns were formed, which were water insoluble and lubricious when wet. The coatings adhered well to the catheter under wet and dry conditions and were sufficiently flexible so as not to crack or peel upon flexing of the catheter.

### Example 20

A SKATER polyurethane drainage catheter was spray coated with Coating Solution AN and dried in an oven for 60 minutes at 75-80° C.

Water insoluble coatings having a uniform thickness between about 7-15 microns were formed, which were water insoluble and lubricious when wet. The coatings adhered well to the catheter under wet and dry conditions and were sufficiently flexible so as not to crack or peel upon flexing of the catheter.

### Example 21

A coating solution is described that uses a quickly evaporating solvent mixture that does not damage or degrade polyurethane substrates. The coating may be used to coat medical devices made entirely or partially from polyurethane, including guidewires covered with polyurethane sleeves, catheters, and drainage and feeding tubes.

A nitinol guidewire with a 0.003″ thick polyurethane sheath extruded over it was dip coated with a single layer of Coating Solution X and dried 20 minutes at 85° C.

Adhesion and lubricity of the coating was tested by rubbing the sample surface after immersion in water. The sample showed good wet adhesion and was lubricious when wet. The use of solvents that evaporated quickly (e.g., THF and alcohol) allowed for rapid drying while providing a coating which was smooth and free of surface cracks and distortions.

### Example 22

A hydrophilic coating solution is described that may be used to coat various types of needles and other types of insertable devices that are used for drug administration, tissue/blood sampling, nutrition, and examinations (e.g., biopsy needles, butterfly needles, and implantable ports). The coating may be loaded with one or more anti-infective agents (e.g., acetylsalicylic acid, salicylic acid, triclosan, bronopol, 5-fluorouracil, and the like) to minimize the potential for infection resulting from insertion of the needle or device into the patient.

A stainless steel needle used to implant a continuous glucose monitor is brush coated with Coating Solution Y and dried for 3 minutes at room temperature.

The lubricious coating allowed for easy insertion of the needle through tough tissue using a needle launcher without the need for additional manual maneuvering to achieve complete needle insertion, as was consistently the case with uncoated needles.

### Example 23

Coating solutions are described that may be used to coat medical devices made entirely or partially from PVC or polyurethane, such as feeding tubes, drainage catheters, and polymeric stents. The formulations may be used as an alternative to two-coat systems (e.g., coating systems that include a primer and/or base coat layer and a hydrophilic top coat layer) which incorporate cellulose ester based materials. It was surprising that a one-layer system worked without the use of a cellulose ester as the stabilizing polymer.

A PVC urinary catheter was dip coated with Coating Solution Z and dried for 60 minutes at 75° C. The catheters were dip coated with a hydrophilic polymer solution (Coating Solution AA) and dried for 45 minutes at 85° C.

Coated PVC catheters exhibited good wet adhesion and wet rub resistance and were lubricious when wet. Coatings that included a lesser amount of hydroxyl function acrylic polymer (e.g., Coating Solution AB) did not adhere as well to the substrate but, nevertheless, retained a certain amount of lubricity under wet conditions.

### Example 24

A coating solution is described that may be used to coat medical devices made entirely or partially from PVC, including feeding tubes, drainage catheters, and polymeric stents.

A single layer of Coating Solution AC was coated on a PVC nelaton catheter and dried for 2 hours at 70° C.

The coated catheter was tested for wet lubricity compared to an uncoated nelaton catheter. Coated and uncoated nelaton catheters were wetted in water and inserted into a 9.5 inch long section of silicone tubing, with a few inches of the catheters remaining outside of the silicone tube. The tubes then were wrapped around a 3 cm diameter mandrill and suspended above a bench top. A weighing basket was attached to the protruding end of the catheters, and weights were added into the baskets to determine the weight needed to pull the catheters from the silicone tube.

The uncoated catheter could not be pulled out of the silicone tube, even with 3975 grams added to the basket. The coated catheter was pulled out of the silicone tube with less than 40 grams in the basket. To test the durability of the coating, the coated catheter was reinserted and pulled out of the silicone tube 50 times, and then 100 times. The weight to pull the catheter out was determined after 50 and 100 insertion/removal cycles. After 50 and 100 cycles, the catheter pulled out of the silicone tube with 32.5 grams, demonstrating that the coating remained durable and lubricious even after 100 insertion/removal cycles.

### Example 25

A coating solution is described that may be used to coat medical devices made entirely or partially from polyurethane, including feeding tubes, drainage catheters, central venous catheters, implants for intravenous drug administration and the like.

Polyurethane catheters were dip coated with a single layer of Coating Solution AD and dried in an oven for 30 minutes at 85° C.

Coated catheters exhibited good wet adhesion and wet rub resistance and were lubricious when wet. The formulations may be used as an alternative to two-coat systems (e.g., coating systems that include a primer and/or base coat layer and a hydrophilic top coat layer) that incorporate cellulose ester based materials.

### Example 26

Coatings are described that may be used to coat medical devices made entirely or partially from stainless steel, including guidewires (vascular and non-vascular), epidural needles, biopsy needles, breast localization needles, bone marrow aspiration needles, and stents (e.g., coronary stents or peripheral vascular stents).

A 0.035″ stainless steel mandrill was coated with Coating Solution AE and dried in an oven overnight. The mandrill then was coated with a hydrophilic polymer solution (Coating Solution AF) and dried in an oven overnight.

Coated mandrills exhibited good rub resistance and lubricity under wet conditions without the use of a primer layer.

### Example 27

Coatings are described that may be used to coat medical devices made entirely or partially from stainless steel, including stents (vascular and non-vascular), guidewires, epidural needles, biopsy needles, breast localization needles, and bone marrow aspiration needles. The solvent system was formulated to minimize the amount and use of unnecessary or hazardous solvents and to improve overall biocompatibility of the coated device.

Stainless steel stents were coated with a primer solution (Coating Solution AG) and dried from 30 minutes at 125° C. The stents then were coated with Coating Solution AH and dried for 30 minutes at 125° C. to form an intermediate tie layer. The stents then were coated with a hydrophilic polymer solution (Coating Solution AI) and dried for 60 minutes at 75° C., followed by vacuum drying for 60 minutes at 75° C.

Coatings having a uniform thickness (typically between about 10-40 microns) were formed which were water insoluble and lubricious when wet. Further, the coating adhered well to the struts of the stent and did not crack when stent was flexed.

### Example 28

The coating can help prevent formation of biofilms, i.e., bacterial growths on the surfaces of medical devices. Segments of medical grade aromatic polyether-based polyurethane tubing were coated with a lubricious coating. Critical surface tension (γ_{c}) for each coating was determined from a Zismon plot (liquid surface tension versus cosine of contact angle). Samples were mounted in flow cells for studies of bioadhesion in constant flow of serum seeded with Staphylococcus epidermidis. Scanning electron microscopy (SEM) of samples before and after exposure in the flow cells yielded information about coating quality and bacterial adhesion.

The coating was prepared by first applying a base coat:

| | |
|---|---|
| ¼ SEC. RS NITROCELLULOSE | 2.434% |
| ISOPROPANOL | 1.043% |
| DIBUTYLPHTHALATE | 0.914% |
| CAMPHOR | 0.669% |
| UVINUL M-40 | 0.046% |
| ACETONE | 0.260% |
| BUTYL ACETATE | 2.328% |
| TOLUENE | 1.562% |
| ETHYL ACETATE | 4.544% |
| AROMATIC POLYURETHANE | 3.290% |
| CYCLOHEXANONE | 21.70% |
| BUTANONE | 1.400% |
| BENZYL ALCOHOL | 8.610% |
| ALIPHATIC POLYURETHANE | 2.835% |
| TETRAHYDROFURAN | 48.365% |

followed by a top coat:

| | |
|---|---|
| ETHANOL | 74.316% |
| BUTYROLACTONE | 17.519% |
| PVP | 4.570% |
| ¼ SEC RS NITROCELLULOSE | 0.002% |
| CYCLOHEXANONE | 3.593% |

The uncoated tubing had a γ_{c} of 22±2 mN/m; the coated tubing had a γ_{c} of 24 mN/m. SEM of the uncoated tubing showed cracks and domains in the extruded surfaces. After residing in the flow cell, large, scattered agglomerations of bacterial cells were observed. The coated tubing, however, was very smooth. After residing in the flow cell, the coated tubing showed sheets of cells and cell media (biofilms) sloughing off.

Friction measurements on the coated tubing showed consistent and uniform lubricity with a coefficient of friction more than an order of magnitude lower than the uncoated sample.

Other embodiments are within the scope of the following claims.

1. A medical device comprising a surface coated with a first layer proximal to the surface, the first layer including an ethylene/acrylic acid copolymer.

2. The device of claim 1, wherein the first layer further includes an epoxy resin.

3. The device of claim 1, wherein the device is a blade device.

4. The device of claim 1, further comprising a second layer coated on the first layer, the second layer including an aromatic polycarbonate based polyurethane.

5. The device of claim 4, wherein the second layer further includes a cellulose based polymer.

6. The device of claim 4, further comprising a third layer coated on the second layer, the third layer including a polyvinyl pyrrolidone.

7. The device of claim 6, wherein the third layer further includes a cellulose based polymer.

8. The device of claim 7, wherein the first layer is coated on the surface by dip-coating with a solution comprising the ethylene/acrylic acid copolymer, an epoxy resin, tetrahydrofuran, dimethyl acetamide, anisole, and xylenes, and drying.

9. The device of claim 8, wherein the second layer is coated on the first layer by dip-coating with a solution comprising an aromatic polycarbonate based polyurethane, a nitrocellulose, dimethylacetamide, anisole, methyl ethyl ketone, and n-butanol, and drying.

10. The device of claim 9, wherein the third layer is coated on the second layer by dip-coating with a solution comprising a polyvinylpyrrolidone, a nitrocellulose, 4-butyrolactone, ethanol, benzyl alcohol, cyclohexanone, and isopropanol, and drying.

11. A medical device comprising a surface coated with a first layer proximal to the surface, the first layer including a cellulose based polymer, a urethane, a melamine resin, and a cross-linkable acrylic resin.

12. The device of claim 11, wherein the device is a guidewire.

13. The device of claim 11, further comprising a second layer coated on the first layer, the second layer including a nitrocellulose, a polyvinylpyrollidone, and a plasticizer.

14. The device of claim 13, wherein the plasticizer is a poly(alkylene oxide).

15. The device of claim 14, wherein the urethane is Tycel 7000, the melamine resin is Cymel 248-8, and the cross-linkable acrylic resin is Paraloid AT-746.

16. The device of claim 15, wherein the polyvinylpyrollidone is PVP K90, and the poly(alkylene oxide) is a polyethylene glycol 400.

17. A medical device comprising a surface coated with a first layer proximal to the surface, the first layer being coated on the surface by contacting the surface with a solution comprising ethanol, benzyl alcohol, cyclohexanone, tetrahydrofuran, a nitrocellulose, 4-butyrlactone, and a polyvinylpyrrolidone, and drying.

18. The device of claim 17, wherein the device is a catheter.

19. The device of claim 18, wherein the surface is a thermoplastic polyurethane elastomer.

20. The device of claim 19, wherein the thermoplastic polyurethane elastomer is a pellethane.

21. A medical device comprising a surface coating bonded to a surface of the device, wherein the coating swells when exposed to body fluids; wherein the coating provides a substantial reduction in surface friction after swelling.

22. The medical device of claim 21, wherein the coating comprises multiple layers.

23. The medical device of claim 21, wherein the coating comprises a polyvinylpyrrolidone, a polyvinylpyrrolidone/vinyl acetate copolymer, and a polyethylene glycol.

24. The medical device of claim 23, wherein the coating further comprises a cellulose ester, a polyvinyl chloride, an acrylic polymer or copolymer, a polyurethane, a polyamide polymer, a polyimide polymer, or an epoxy resin.

25. The medical device of claim 23, wherein the polyvinylpyrrolidone has a molecular weight of at least 80 kDa.

26. The medical device of claim 23, wherein the polyvinylpyrrolidone has a molecular weight in the range of 90 kDa to 1,200 kDa.

27. The medical device of claim 23, wherein the cellulose ester is a nitrocellulose.

28. The medical device of claim 23, wherein the device is selected from the group consisting of a catheter, an arterial catheter, a short-term central venous catheter, a long-term central venous catheter, a peripheral venous catheter, a vascular port catheter, a dialysis device, a guide wire, an introducer, a knife, a needle, an amniocentesis needle, a biopsy needle, an infusion needle, an introducer needle, a suture needle, an obdurator, a pacemaker, a pacemaker lead, a penile prosthesis, a scalpel, a shunt, an arteriovenous shunt, a hydrocephalus shunt, a stent, a biliary stent, a coronary stent, a neurological stent, a urological stent, a vascular stent, a syringe, a trocar, a tube, a drain tube, an endotracheal tube, a gastroenteric tube, a nasogastric tube, an intermittent urinary catheter, a Foley catheter, a long-term urinary device, a tissue bonding urinary device, a urinary dilator, a urinary sphincter, a urethral inserts, and a wound drain.

29. The device of claim 21, wherein the surface coating includes a first layer proximal to the device surface, the first layer including an ethylene/acrylic acid copolymer.

30. The device of claim 29, wherein the first layer further includes an epoxy resin.

31. The device of claim 29, wherein the device is selected from the group consisting of a catheter, an arterial catheter, a short-term central venous catheter, a long-term central venous catheter, a peripheral venous catheter, a vascular port catheter, a dialysis device, a guide wire, an introducer, a knife, a needle, an amniocentesis needle, a biopsy needle, an infusion needle, an introducer needle, a suture needle, an obdurator, a pacemaker, a pacemaker lead, a penile prosthesis, a scalpel, a shunt, an arteriovenous shunt, a hydrocephalus shunt, a stent, a biliary stent, a coronary stent, a neurological stent, a urological stent, a vascular stent, a syringe, a trocar, a tube, a drain tube, an endotracheal tube, a gastroenteric tube, a nasogastric tube, an intermittent urinary catheter, a Foley catheter, a long-term urinary device, a tissue bonding urinary device, a urinary dilator, a urinary sphincter, a urethral inserts, and a wound drain.

32. The device of claim 29, wherein the surface of the device comprises a metal or metal alloy.

33. The device of claim 32, wherein the metal or metal alloy is gold, nitinol, nickel, platinum, stainless steel, tantalum, or titanium.

34. The device of claim 21, wherein the surface of the device comprises a polymer or copolymer selected from a silicone, a polyethylene, a polypropylene, a polyester, a polytetrafluoroethylene, a polyamide, a polyimide, and a styrene/isobutylene copolymer.

35. The device of claim 29, further comprising a second layer coated on the first layer, the second layer comprising a polyurethane, a poly(vinyl chloride), a polyamide, an acrylate polymer or copolymer, a polyimide, a polyester, a polycarbonate urethane, an aliphatic urethane, an aromatic urethane, or a cellulose ester.

36. The device of claim 29, further comprising a third layer coated on the second layer, the third layer including a polyvinylpyrrolidone, a polyethylene glycol, a polyethylene oxide, or a polyvinylpyrrolidone/vinyl acetate copolymer.

37. The device of claim 36, wherein the third layer further comprises a cellulose ester, a polyamide, an acrylic polymer/copolymer, an epoxy resin, a melamine resin, a formaldehyde resin, a urethane, or a cross-linkable acrylic resin.

38. The device of claim 37, wherein the cellulose ester comprises cellulose nitrate, and the urethane comprises aliphatic urethanes, aromatic urethanes and polycarbonate urethanes.

39. A medical device having a surface coated with a first layer proximal to the surface, the first layer comprising a cellulose ester, a urethane, a melamine resin, a formaldehyde resin, and a cross-linkable acrylic resin.

40. The device of claim 39, further comprising a second layer coated on the first layer, the second layer comprising a nitrocellulose, an aliphatic urethane, an aromatic urethane and a polycarbonate urethane.

41. The device of claim 39, wherein the device is selected from the group consisting of a catheter, an arterial catheter, a short-term central venous catheter, a long-term central venous catheter, a peripheral venous catheter, a vascular port catheter, a dialysis device, a guide wire, an introducer, a knife, a needle, an amniocentesis needle, a biopsy needle, an infusion needle, an introducer needle, a suture needle, an obdurator, a pacemaker, a pacemaker lead, a penile prosthesis, a scalpel, a shunt, an arteriovenous shunt, a hydrocephalus shunt, a stent, a biliary stent, a coronary stent, a neurological stent, a urological stent, a vascular stent, a syringe, a trocar, a tube, a drain tube, an endotracheal tube, a gastroenteric tube, a nasogastric tube, an intermittent urinary catheter, a Foley catheter, a long-term urinary device, a tissue bonding urinary device, a urinary dilator, a urinary sphincter, a urethral inserts, and a wound drain.

42. The device of claim 39, wherein the surface comprises a metal or a metal alloy.

43. The device of claim 42, wherein the metal or metal alloy is gold, nitinol, nickel, platinum, stainless steel, tantalum, or titanium.

44. A method of making a medical device, comprising forming a first layer on a surface of the device, the first layer including an ethylene/acrylic acid copolymer.

45. The method of claim 44, further comprising forming a second layer on the first layer, the second layer including an aromatic polycarbonate based polyurethane.

46. The method of claim 45, further comprising forming a third layer on the second layer, the third layer including a polyvinyl pyrrolidone.

47. The method of claim 44, wherein forming the first layer includes contacting the surface of the device with a solution comprising the ethylene/acrylic acid copolymer, an epoxy resin, tetrahydrofuran, dimethyl acetamide, anisole, and xylenes, and drying.

48. The method of claim 44, wherein forming the second layer includes contacting the first layer with a solution comprising an aromatic polycarbonate based polyurethane, a nitrocellulose, dimethylacetamide, anisole, methyl ethyl ketone, and n-butanol, and drying.

49. The method of claim 44, wherein forming the third layer includes contacting the second layer with a solution comprising a polyvinylpyrrolidone, a nitrocellulose, 4-butyrolactone, ethanol, benzyl alcohol, cyclohexanone, and isopropanol, and drying.
